(19) United States
(12) Patent Application Publication
(10) Pub. No.: US 2009/0311236 A1
(43) Pub. Date: Dec. 17, 2009

(54) Therapeutic Peptide Compositions And Methods Of Making And Using Same

(75) Inventors: Bonawentura Kochel, Wroclaw (PL); Patrick J. Offman, Northville, MI (US)

Correspondence Address: CARRIER BLACKMAN AND ASSOCIATES, 43440 WEST TEN MILE ROAD, EATON CENTER, NOVI, MI, 48375, US

(73) Assignee: IMMUNE @WORK, INC., Rosemead, CA (US)

(21) Appl. No.: 12/137,236

(22) Filed: Jun. 11, 2008

(51) Int. Cl. A61K 38/48; A61K 38/47

(52) U.S. Cl. 424/94.61; 424/94.63; 424/94.6

(57) ABSTRACT

Therapeutic compositions include peptide formulations having a mixture of serum, albumin, casein, peptone, pacreatin, and sodium hydroxide. The peptide compositions do not include nucleic acid or any derivatives thereof. The peptide compositions have beneficial therapeutic properties to boost human immune system and to treat various ailments including malaria, allergy and inflammation. The peptide compositions, one having a molecular weight greater than 10 kDa and having amino acid residues in a range of 27 and 315, and the other having a molecular weight equal to or less than 10 kDa and amino acid residues in a range of 27 and 54, their structures and therapeutic properties are discussed.

### BACKGROUND OF THE INVENTION

1. Field of the Invention

The present invention relates therapeutically beneficial peptide compositions, which may be used as antiviral agent and as an agent useful in treating auto immune diseases. More particularly, the present invention relates to such peptide compositions which involve no nucleic acid components, are non-toxic, and may be administered in various manners including as food supplements. The present invention also related to methods of preparing the peptide compositions and various useful forms of the peptide compositions, and methods of using the peptide compositions and the various useful forms thereof.

2. Description of the Related Art

In the art there are several known compositions containing peptides and nucleic acids which have been used for treating various ailments including viral infections, immune system diseases, etc. One such composition, distributed under the trademark Reticulose®, which has been used as antiviral agent for humans in relation to treatment of viral infections/diseases such as influenza, herpes, infectious mononucleosis, hepatitis A and B, and HIV. Reticulose®, also generally referred to as nucleophosphoprotein and a lipoprotein nucleic acid solution, was originally conceived by Dr. Vincent LaPenta around 1934 and was commercially available in the U.S. for a period ending in 1962.

Reticulose® is known to be formulated through a mixture of casein, beef peptone, ribonucleic acid (RNA), beef serum (blood) albumin, sodium hydroxide and distilled water which is processed through heat, pressurization and filtration to a solution that is of such a small molecular weight as to be compatible with any human blood type, as discussed in U.S. Pat. No. 5,849,196, for example. Essentially, the conventional Reticulose® composition is a complex solution of peptides and nucleic acids in which nucleic acid fragments are associated or possibly associated with short chain peptides, and wherein the molecular weight of the active components ranges from approximately 1 to 25 kDa.

Also very significantly, the conventional composition has been shown to be substantially free from side effects and systemic toxicity, unlike many other antiviral agents, including AZT and beta interferon. Other background information relating to Reticulose® is presented in U.S. Pat. No. 5,849,196.

Over the past 10-15 years there have been disclosed several other peptide-nucleic acid compositions which are essentially improvements to the conventional Reticulose® composition, including some improvements conceived by one of the present inventors, B. Kochel. Examples of these other peptide-nucleic acid compositions, and methods of making and using same are disclosed in U.S. Pat. Nos. 5,807,839; 5,807,840; 5,849,196; 5,902,786; 6,268,349; 6,303,153; 6,312,602; 6,352,226; 6,440,658; 6,528,098; 6,670,118; 6,696,422; 6,921,542; 7,067,139; and 7,074,767.

As disclosed in these patents, each of the known compositions is formulated from substantially the same disclosed starting ingredients, which are matters of public knowledge, but then the ingredients are processed in various manners, e.g., fractionated according to weight, to isolate and obtain various components, which are then selectively used for treating various afflictions.

Some of the known compositions are identified under various trade names, e.g., Product R, but like the conventional Reticulose® composition, all of these other known compositions require nucleic acid as an essential ingredient. The entire disclosures of each of the above-identified patents are incorporated herein by reference.

Several uses for the peptide-nucleic acid compositions—such as treatment of HIV infections, autoimmune disease, topical treatment of skin disease, B19 parvovirus infections, treatment of basal cell carcinoma, treatment of canine distemper, simulation of red blood cell production, etc.—have been disclosed in these patents, as well as in other publications.

Although the known peptide-nucleic acid compositions are useful for their intended purposes, these compositions still remain to be improved upon in terms of effectiveness for treating various ailments including viral infections and immune system diseases. Also, the known methods for preparation and use of the known compositions remain to be improved upon in terms of cost, efficiency, and effectiveness. Research program leading to the present invention was initiated in 2006.

### SUMMARY OF THE INVENTION

The present invention has been developed to fulfill the above-discussed desiderata.

Specifically, after substantial research into the matter, applicant has determined that the nucleic acid components of the known compositions do not appear to provide any significant therapeutic effect, and may even inhibit some of the therapeutic effects of the peptide components of the known compositions.

Relatedly, applicant has also determined that beneficial therapeutic peptide compositions may be prepared using ingredients and procedures such as those disclosed in the above-identified patents, but excluding all ingredients containing nucleic acids or derivatives thereof. Research shows that thus obtained peptide compositions, without any nucleic acid components, are compositionally distinct from the known peptide-nucleic acid compositions, and possess advantageous characteristics not possessed by the known compositions.

It is an object of the present invention to provide a composition containing peptides which provides a mainly stimulatory effect, and another such composition which provides a mainly inhibitory effect on phagocystic activity of neutrophils in humans, and which otherwise has substantially no toxicity or ill side effects associated therewith.

It is another object of the present invention to provide a relatively simple method of preparing stable forms of the compositions, including solutions, ointments, and food supplements.

It is a further object of the invention to provide protocols for treating various viruses in humans and other animals using the various forms of the compositions.

It is yet another object of the invention to provide an improved composition containing peptides, but without any nucleic acid components, which is tailored or modified to treat different viruses, auto immune diseases and the like.

According to a first aspect of the present invention, there is provided a bioactive peptide composition formed from a mixture consisting essentially of serum, albumin, casein, peptone, a digestive enzyme, and a base. The digestive enzyme may comprise one or more of the major digestive enzymes of the human body such as: a pancreatin supplying amylase, protease, lipase, protease II, or protease III, aylase, lactase, lipase, and combinations thereof. The base may comprise sodium hydroxide. Also, the composition may be orally administered, e.g., as a food supplement.

As will be understood, no nucleic acid component(s) are included in the mixture consistent with applicant's findings. Further, tests conducted by applicant show that the bioactive peptide composition of the present invention has desirable properties which have not been shown or indicated for the known peptide-nucleic acid compositions. These properties include enhanced free radical scavenging properties, enhanced antioxidative properties, enhanced enzymatic activity of SOD in human blood and of GPx in human erythrocytes, and an antimalarial function.

The mixture used in formulation of the bioactive peptide composition may include the following relative percentages of components: for every 50 ml serum, 1-2 g albumin; 10-50 g casein; 10-30 g peptone; 0.1-0.1 g digestive enzyme; and 5-10 g base.

The mixture used in formulation of the bioactive peptide composition may be separated into fractions based on molecular weight, and the different fractions may be used for treating different afflictions. For example, the formulation may be separated into two fractions, e.g., a first fraction or composition including molecules having a molecular weight greater than 10 kDa, and a second fraction or composition including molecules having a molecular weight of 10 kDa and lower. Applicant has found that such a first fraction including molecules with a molecular weight of at least 10 kDa comprises peptides without aromatic portions, and will stimulate a phagocytic activity of neutrophils when administered a dose above a predetermined quantity of a formulation.

Such formulation containing the first fraction is effective or is expected to be effective in treating diseases or illnesses as a result of a weakened or suppressed immune system, such diseases in this category are AIDS, Leukemia, Hepatitis, Flu, Mononucleosis, Epstein-Barr Syndrome, Cancer, Malaria, Herpes, and Non-Hodgkins Lymphoma. A formulation containing the second fraction is effective or expected to be effective in treating auto immune diseases or disorders. Diseases included in the auto immune disorders are Allergies, Asthma, Lupus, and Scleroderma. Clinical observations also indicated positive benefits with Rheumatoid Arthritis and Multiple Sclerosis. Thus, the active peptide components of the bioactive peptide composition may be segregated generally according to molecular weight and the different resulting groups of components may be selectively used to treat different viruses and auto immune diseases accordingly.

Applicant has determined that while the molecular mass of molecules in the first fraction is generally above 10 kDa, and evaluation of the first fraction using chromatographic bands recorded by SDS-PAGE electropherosis shows that the molecular mass of the largest molecules in the fraction is approximately 35 kDa. Further, although applicant has sought to achieve a fine separation between the first and second fractions at approximately 10 kDa, invariably the separation is not perfect and the first fraction may contain small amounts of lower weight molecules, e.g., the molecular mass of the smallest molecules in the first fraction may be approximately 3 kDa. The first fraction may have a protein content of about 10.5 μg/μg obtained at absorbance at 280 nm, and is about 2.5 μg/μg according to bicinchoninic acid assay method.

Further, the number of amino acid residues in the first fraction may be in a range of 27 and 315, while exemplary purified peptide compositions of sequences according to the second fraction include the following.

Sequences having 59 amino acid residues such as GlyGluProGlyAlaAspLysSerThrProMetPheValLeuGlnHisArgProlleGluThrGlySerLysTyrGluAlaAspArg-PheGlnProValAlalleGlyAsnLysArgGlnAlaLeuGlnGlyProValGluArgLeuGluLysProGlyPheTyrLeuProGlnLys, GlyGluProGlyAlaAspLysSerThrProMetPheValLeuGlnHisArgProlleGluThrGlySerLysTyrGluAlaAspArg-PheGlnProValAlalleGlyAsnLysArgGlnAlaLeuGlnGlyProValGluArgLeuGluLysProGlyPheTyrLeuProGlnGlu, GlyGluProGlyAlaAspLysSerThrProMetPheValLeuGlnHisArgProlleGluThrGlySerLysTyrGluAlaAspArg-PheGlnProValAlalleGlyAsnLysArgGlnAlaLeuGlnGlyProValGluArgLeuGluLysProGlyPheTyrLeuProGlnAsn, AlaGluProGlyAlaAspLysSerThrProMetPheValLeuGlnHisArgProlleGluThrGlySerLysTyrGluAlaAspArg-PheGlnProValAlalleGlyAsnLysArgGlnAlaLeuGlnGlyProValGluArgLeuGluLysProGlyPheTyrLeuProGlnAsn, and other similar peptide sequences in which the terminal residues are varied, and in which the 6^{th}-59^{th} residues can be ordered randomly.

Applicant has determined that while the molecular mass of molecules in the second fraction is generally 10 kDa or lower and wherein molecular mass of peptide components in the second fraction, evaluated by a using chromatographic bands recorded by SDS-PAGE electropherosis, generally ranges between 3 kDa and 6 kDa.

The second fraction may have a protein content of about 8 μg/μg obtained at absorbance at 280 nm; and may be about 1.5 μg/μg according to bicinchoninic acid assay method.

Further, the number of amino acid residues in the second fraction may be in a range of 27 and 54, while exemplary purified peptide compositions of sequences according to the second fraction include:

Sequences having 27 amino acid residues such as GlyGluProAlaProValLeuGlyGlnAlaValPheProSerThrLeuGluLysArgTyrIleAspArgGlyGlnAsnLys, GlyGluProAlaProValLeuGlyGlnAlaValPheProSerThrLeuGluLysArgTyrIleAspArgGlyGlnAsnGlu, GlyGluProAlaProValLeuGlyGlnAlaValPheProSerThrLeuGluLysArgTyrIleAspArgGlyGlnLysAsn, AlaGluProGlyProValLeuGlyGlnAlaValPheProSerThrLeuGluLysArgTyrIleAspArgGlyGlnAsnGlu, and other similar peptide sequences in which the terminal residues are varied, and in which the 6^{th}-27^{th} residues can be ordered randomly.

Sequences having 41 amino acid residues such as

### AlaProGlyAlaProPheAsnLysValThrGluMetLeuAspArgTyrSerIleGlyGlnHisGluArgValProLeuGlyPheGln-GluIleLysSerGlyProGluLysGlnArgGlnLeu,

### AlaProGlyAlaProPheAsnLysValThrGluMetLeuAspArgTyrSerIleGlyGlnHisGluArgValProLeuGlyPheGln-GluIleLysSerGlyProGluLysGlnArgGlnLys,

### AlaProGlyAlaProPheAsnLysValThrGluMetLeuAspArgTyrSerIleGlyGlnHisGluArgValProLeuGlyPheGln-GluIleLysSerGlyProGluLysGlnArgGlnGlu,

### GlyProGlyAlaProPheAsnLysValThrGluMetLeuAspArgTyrSerIleGlyGlnHisGluArgValProLeuGlyPheGln-GluIleLysSerGlyProGluLysGlnArgGlnGlu,

and other similar peptide sequences in which the terminal residues are varied, and in which the 6^{th}-27^{th} residues can be ordered randomly.

The second fraction, at 16.5% volume/volume concentration, may be administered to humans to enhance activity of superoxide dismute and glutathione peroxide in human blood cells.

According to a second aspect of the present invention, there is provided a bioactive peptide composition formed from a mixture consisting essentially of: serum; albumin; casein; peptone; a digestive enzyme; and a base, wherein said peptide composition includes peptide bands which are stainable with Coomassie Blue, and which are not stainable with silver ions during SDS-PAGE electrophoresis analysis.

Again, the digestive enzyme may be one or more of the major digestive enzymes of the human body such as: a pancreatin supplying amylase, protease, lipase, protease II, or protease III, aylase, lactase, lipase, and combinations thereof, while the base may comprise sodium hydroxide. Other objects, advantages and salient features of the invention will be apparent from the following detailed description which, in conjunction with the annexed drawings, discloses presently illustrative embodiments of the invention.

### BRIEF DESCRIPTION OF THE DRAWINGS

FIG. 1 is a representation of results of gel filtration results of the first fraction sample (7.5 mg of total protein was loaded) performed on TSK column.

FIG. 2 is a representation of results of gel filtration of the second fraction sample (4.5 mg of total protein was loaded) performed on TSK column.

FIG. 3 is representation of result of SDS-PAGE electrophoresis, of fractions from TSK column for the first fraction of FIG. 1, in which every two samples were analyzed and the fractions are denoted by retention times.

FIG. 4 is representation of result of SDS-PAGE electrophoresis, of fractions from TSK column for the second fraction of FIG. 2, in which every two samples were analyzed and the fractions are denoted by retention times.

FIG. 5 shows results of HPLC chromatography on a C-18 column of pooled fractions 57-65 from gel filtration of the first fraction sample of FIG. 1.

FIG. 6 shows results of SDS-PAGE electrophoresis of resulting fractions 57-65 from gel filtration of the first fraction sample (FIG. 3) at 220 nm and 280 nm.

FIG. 7 shows 1^{st}- and 2^{nd}-order structure of Peptide 1.

FIG. 8 shows 1^{st}- and 2^{nd}-order structure of Peptide 2.

FIG. 9 shows 1^{st}- and 2^{nd}-order structure of Peptide 3.

FIG. 10 shows 1^{st}- and 2^{nd}-order structure of Peptide 4.

FIG. 11 shows 1^{st}- and 2^{nd}-order structure of Peptide 5.

FIG. 12 shows 1^{st}- and 2^{nd}-order structure of Peptide 6.

FIG. 13 shows 1^{st}- and 2^{nd}-order structure of Peptide 7.

FIG. 14 shows 1^{st}- and 2^{nd}-order structure of Peptide 8.

FIG. 15 shows 1^{st}- and 2^{nd}-order structure of Peptide 9.

FIG. 16 shows 1^{st}- and 2^{nd}-order structure of Peptide 10.

FIG. 17 shows 1^{st}- and 2^{nd}-order structure of Peptide 11.

FIG. 18 shows 1^{st}- and 2^{nd}-order structure of Peptide 12.

FIG. 19 is plot showing amino acid analysis, ion-exchange chromatograms of acid hydrolysates of sample of the first fraction.

FIG. 20 is plot showing amino acid analysis, ion-exchange chromatograms of acid hydrolysates of sample of the second fraction.

FIG. 21 is a schematic reaction of cyclic nitrone spin trap (BMPO) with a volatile radical (R*) that leads to the formation of stable paramagnetic nitroxide spin adduct BMPO*-R detectable by electron paramagnetic resonance spectroscopy (unpaired electron is localized on the pi-orbital between nitrogen and oxygen atom of N—O moiety on BMPO adduct).

FIG. 22 is an EPR spectrum of BMPO*-OH adduct (Left), and the corresponding spectrum simulated at the assumption of the existence of two diastereoisomers (Right).

FIG. 23 is an EPR spectra of hydroxyl radical adduct with BMPO measured in the absence (A) and presence of second fraction (B) or first fraction (C) at the final concentration of 26 mg/ml. Magnified region of the first lines is shown in the graph (D) (The dashed line indicates the component coming from the residual carbon-centered radical).

FIG. 24 is an EPR spectrum of carbon-centered radical adduct with BMPO spin trap (left); the simulated EPR spectrum was performed, assuming the existence of two diastereoisomers (right).

FIG. 25 is an EPR spectra of carbon centered radical adduct with BMPO spin trap generated by Fenton reaction in the presence of 23% of ethanol (A); in the same conditions, signals measured with addition of second fraction (B) and first fraction (C); and the first two lines for spectra A, B and C are magnified and presented in the panel D.

FIG. 26 is EPR spectrum of azide radical adduct with BMPO (left); the corresponding simulation of the spectrum (right) was performed assuming the presence of 88% of azide radical (aN=13.96 G, aN2=3.15 G, aH=12.7 G) and 12% of hydroxyl radical.

FIG. 27 is an EPR spectra of azide radical adduct with BMPO spin trap, generated in Fenton reaction in the presence of 250 mM of NaN₃; the control sample was measured in the absence of the peptides (A); the EPR spectra of azide radical trapped by BMPO in the presence of the first fraction or the second fraction in concentration of 26 mg/ml (B and C respectively); the first three lines of EPR spectra measured in the absence of the peptides, in the presence of the first fraction and the second fraction are juxtaposed in the graph (D).

FIG. 28 is an EPR spectra of a radical that is generated by the reduction of cumene hydroperoxide by Fe²⁺ ions; the radical trapped by BMPO represents essentially the same shape as in the case of carbon-centered radical; the control sample gives the signal shown in the panel A; EPR signal recorded after addition of the first fraction and the second fraction is presented in the panels B or C, respectively; and the first EPR lines measured for all three samples are presented in the panel D.

FIG. 29 is an EPR signal of BMPO adduct with the radical products generated by spontaneous decomposition of cumene hydroperoxide catalyzed by trace amount of transition metals (A); EPR signal measured under the same conditions but in the presence of the second fraction (B) and the first fraction (C) at concentration of 26 mg/ml of each.

FIG. 30 is an EPR spectrum of superoxide adduct with BMPO spin trap (Black line); the corresponding simulation of the spectrum (Red line) was performed using parameters: aN=13.368 G, aH=11.049 G.

FIG. 31 is an EPR spectrum of nitroxide created by oxidation of CPH by one electron oxidants (superoxide, alkoxyl radical, ferricyanide ions, etc.); Hyperfine structure comes exclusively from interaction of electron spin with the nearest nitrogen nuclei (I=1), irrespective of the type of oxidant.

FIG. 32 is an oxidation of CPH hydroxylamine by superoxide radical generated by X/XO system in the absence (closed symbols) or the presence of 200 U/ml of SOD (open symbols) (A); Oxidation rate measured for control (circle), in the presence of second fraction (square) and first fraction (triangle) after subtraction of the background oxidation of CPH measured in the presence of SOD.

### DETAILED DESCRIPTION

The detailed description of the present invention is provided with respect to contents of the peptide preparations and their physical/chemical and biological functions through in vitro experiments on human biological materials, and biological functions through in vivo experiments on animals.

### Part 1: Preparation of Peptide Compositions and Contents Thereof

### I. Preparation of Peptide Compositions of the Present Invention

A laboratory scale preparation of peptide compositions of the present invention is discussed below. A full scale preparation of the peptide compositions may be performed by up-scaling the amounts (masses and volumes) of the raw material provided hereinbelow, by multiplying each by an arbitrary factor 39.846.

First, a 75.3 ml of distilled water filled in a 0.5 L glass flask was bubbled by argon for several minutes prior to addition of the raw materials.

Next, when the distilled water was sufficiently bubbled by argon, a predetermined quantities of the following raw materials (compounds), shown in Table 1, were added in according a predetermined sequence (i.e., each compound was added after complete dissolution of the previous one) in the glass flask filled with the distilled water.

TABLE 1
Raw Materials for Peptide Compositions

| Sequence of Adding | Raw Material | Amount | |
|---|---|---|---|
| 1 | newborn calf serum | 50 | ml |
| 2 | albumin | 1.506 | g |
| 3 | casein | 25 | g |
| 4 | peptone | 15.06 | g |
| 5 | digestive enzyme | 0.301 | g |

The reaction was performed in the glass flask, continuously purged by a stream of argon. The flask was mounted on a magnetic stirrer and the compounds (raw materials) were added using a plastic funnel.

The solution was mixed approximately for 2 hrs, then 7.53 g of NaOH was added and the solution was mixed for further 0.5 hr. After this period pH of the mixture was adjusted to 8.5 using about 150 ml of 1 M HCl.

The flask was then autoclaved for approximately 4 hours at 125° C. After autoclaving, the flask was cooled at room temperature, pH of the mixture was adjusted to 7.5 using about 5 ml of 3 M HCl, the solution was centrifuged for 20 minutes at 3500 g and then filtered using 0.1 μm filter (e.g., Millipak-20 disposable filter unit obtained from Millipore Corp., USA). After filtering the mixture, a total volume of the mixture was 212 ml.

Distilled water was added in the filtered solution so as to make its final volume about 3012 ml. The solution was then purged with argon and again autoclaved for 1 hour at 125° C. The solution (also referred as hydrolyzate) was then cooled at room temperature and filtered through a 10 kDa cut-off filter (e.g, PLGC regenerated cellulose membrane obtained from Milliopre Corp., USA) until 50% of the starting volume, e.g. 50% of 3012 ml, was reached.

The high molecular mass filtrate, i.e., the condensed solution with molecules having molecular weight greater than 10 kDa is called a “first composition” or “first fraction”. The low molecular mass filtrate, i.e., the solution with molecules having molecular weight less than or equal to 10 kDa was called a “second fraction” or “second fraction”.

### II. Peptide Contents of the Peptide Compositions

### (i) Protein Estimation

Total protein content of the peptide compositions was estimated by UV absorbance measurement (absorbance coefficient at 280 nm was assumed to be 1 at 1 mg/ml) and by bicinchoninic acid assay (BCA) method calibrated on bovine serum albumin (BSA).

The results of total protein content measurements for the first fraction and the second fraction are presented in the following Table 2. These measurements indicate concentration of total protein obtained from hydrolyzates of the peptide compositions.

TABLE 2
Concentration of total protein obtained
from protein hydrolyzates of the peptide compositions.

Protein content (μg/μl)

| Peptide composition | Absorbance at 280 nm | BCA assay |
|---|---|---|
| First Fraction | 10.5 | 2.5 |
| Second Fraction | 8.0 | 1.5 |

### (ii) Gel Filtration

Chromatography was performed on the peptide compositions using Dionex P680 HPLC system (Dionex, USA) equipped with TSKGel G2000SW 2.15×30 cm (Tosoh, Japan) column. Separations were conducted on each of the peptide compositions in 0.02 M ammonium acetate buffer pH 5.5 at 1.8 ml/min flow rate. The data were collected using spectrophotometric detection at 220 and 280 nm.

In order to minimize loss of potentially active peptide, a peptide purification procedure was performed on whole lyophilized samples, without desalting steps. A 10-ml portions of both hydrolyzates were lyophilized, obtained dry samples, and were redissolved in 1 ml of water. The concentrated solutions were loaded on the TSK gel filtration column in 300-μl portions.

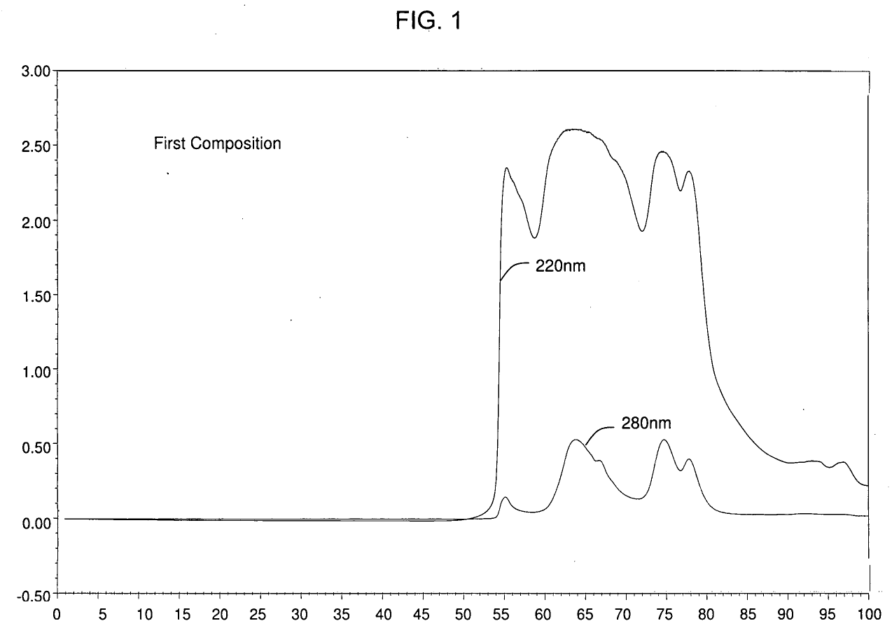

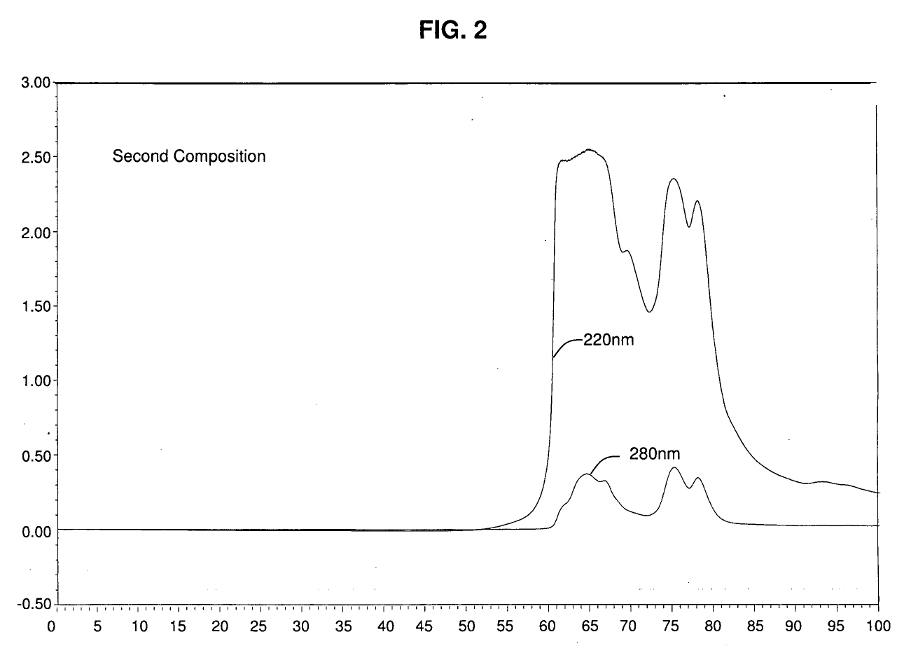

The above 300-μl amounts contained 7.5 mg of total protein (according to BCA assay) in case of the first fraction sample and 4.5 mg of total protein in case of the second fraction sample. FIGS. 1 and 2 show the chromatograms for the first fraction and the second fraction, respectively.

In other words, FIG. 1 is a representation of results of gel filtration results of the first fraction sample (7.5 mg of total protein was loaded) performed on TSK column; and FIG. 2 is a representation of results of gel filtration of the second fraction sample (4.5 mg of total protein was loaded) performed on TSK column.

(iii) Denaturing Gel Electrophoresis (SDS-PAGE)

Electrophoresis was performed on the peptide compositions under reducing conditions using tris-tricine peptide-separating gels (as described in Anal. Biochem. 166 (1987) 368-379) in MiniProtean II apparatus (obtained from BioRad, USA). After electrophoresis, the gels were fixed in 50% methanol, 10% acetic acid and then subjected to staining by Coomassie Brillant Blue R-250 or by silver ions.

The peptide composition samples were collected during gel filtration and 1.8-ml fractions thereof were lyophilized, redissolved in water and subjected to SDS-PAGE electrophoresis. Resulting gels were fixed and stained with Coomassie Brillant Blue R-250 and with silver ions.

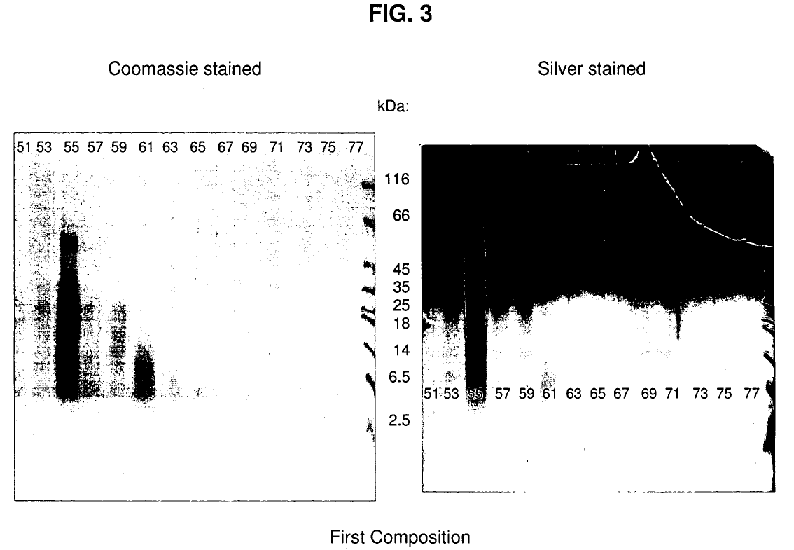

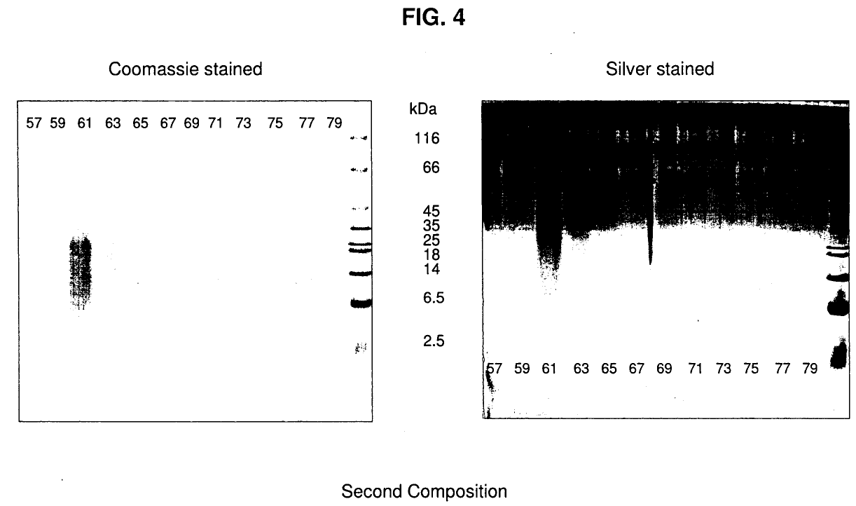

The electrophoregrams obtained for the first fraction and the second fraction are presented on FIGS. 3 and 4, respectively. In other words, FIG. 3 is a representation of results of SDS-PAGE electrophoresis, of fractions from TSK column for the first fraction of FIG. 1, in which every second sample was analyzed and the fractions are denoted by retention times; and FIG. 4 is representation of result of SDS-PAGE electrophoresis, of fractions from TSK column for the second fraction of FIG. 2, in which every second sample was analyzed and the fractions are denoted by retention times.

From FIGS. 3 and 4, it can be seen that low molecular mass components (both Coomassie- and silver-stainable) are visible only in case of the first fraction samples (FIG. 3), in fractions 57-65 (especially in fraction 61). These compounds forms a distorted cushiony bands, too diffuse to direct analysis by sequencing from a PVDF membrane.

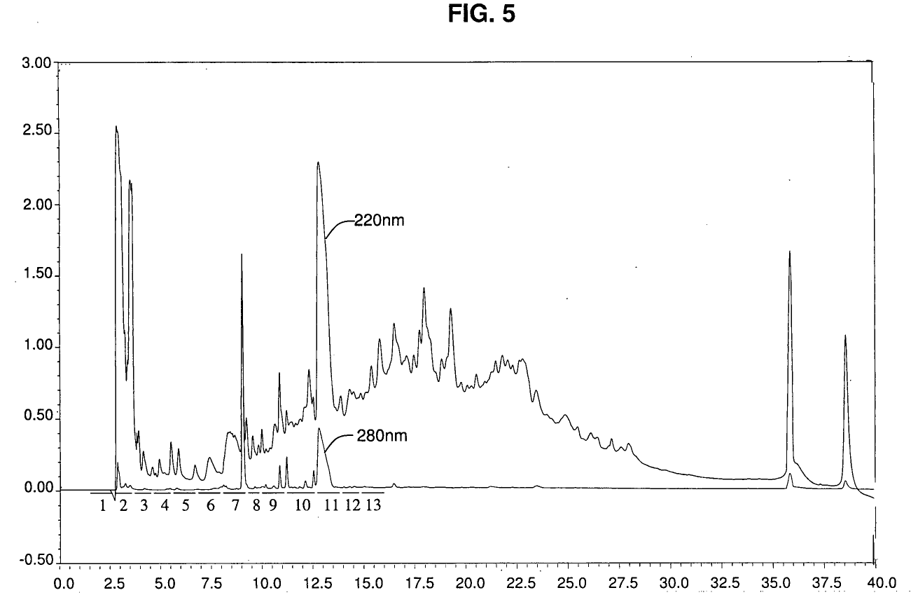

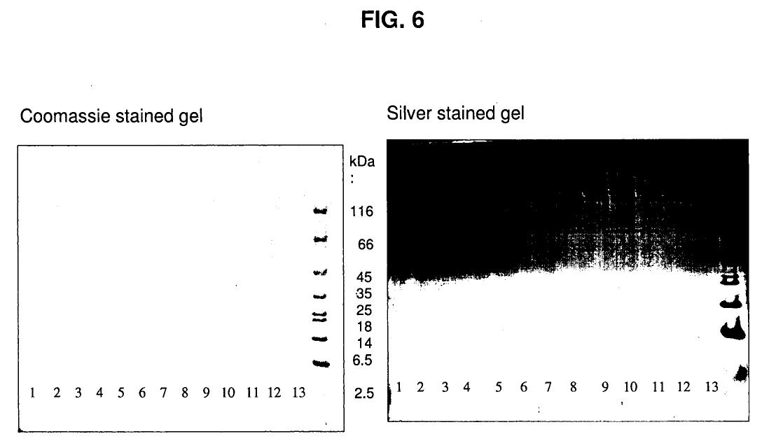

For better separation of those compounds, the fractions 57-65 were pooled, lyophilized, redissolved in 0.1% TFA and subjected to HPLC separation on a C-18 column (FIG. 5). The manually collected fractions from this chromatography were lyophilized, dissolved in water and analyzed by SDS-PAGE (FIG. 6).

It can be seen form FIG. 5 that the analyzed mixture gives rich and relatively well resolved chromatogram, comprising peaks that absorb both 220 and 280 nm light (especially fractions 1-6). Such results suggest that these compounds are irreversibly adsorbed on the used C-18 HPLC column.

### (iv) Length and Number of Peptides of the Peptide Compositions

The peptides of the first fraction and the second fraction were further characterized with regard to the number of amino acid residues in a single peptide, and the number of different peptides.

The following values of peptide's molecular masses (M) for the second fraction and the first fraction were determined using the chromatographic bands recorded by SDS-PAGE electrophoresis.

first fraction: lower limit for M=3000 Da
upper limit for M=35000 Da
Mat the band maximum=10000 Da
   second fraction: lower limit for M=3000 Da,
upper limit for M=6000 Da,
M at the band maximum=4500 Da.

Amino acid sequencing of peptides of the first and the second fractions was conducted, using the Edman degradation, to determine the first five positions of amino acids in peptides (beginning from the N-terminal) in two Fractions, 3 and 5. The amino acid residues in these positions were found identical in the both fractions. Amino acids for which amounts were found to be greater than 50 pmole are shown in the following Table 3, in which, nᵢ is the number of amino acid residuals occupying the i^{th} position in the peptides.

TABLE 3
The first five amino acid positions in peptides of the second fraction
and the first fraction
Amino acid residue's position in peptides

| 1^{st} | 2^{nd} | 3^{rd} | 4^{th} | 5^{th} |
|---|---|---|---|---|
| Gly | Glu | Pro | Gly | Pro |
| Ala | Pro | Gly | Ala | Ala |
| Val | Val | | His | Gly |
| | Leu | | | |
| | Gly | | | |
| | Ala | | | |
| | Phe | | | |
| n₁ = 3 | n₂ = 7 | n₃ = 2 | n₄ = 3 | n₅ = 3 |

Relative contents of amino acids in the first and second fractions (x_{B}, x_{P}) are expressed in percentage by weight in the following Table 4.

TABLE 4
Molecular masses of amino acid residues and their relative contents
in the first and second fractions

| | Molecular mass | Relative contents of | Relative contents of |
|---|---|---|---|
| | of amino acid | amino acids in the | amino acids in the |
| Amino acid | residue | first fraction | second fraction |
| AA | m_{AA} [Da] | x_{AA} [%] | x_{AA} [%] |
| Gly | 57.05 | 9.75 ± 0.32 | 9.46 ± 0.12 |
| Ala | 71.08 | 6.16 ± 0.10 | 5.90 ± 0.06 |
| Ser | 87.08 | 4.00 ± 0.01 | 3.87 ± 0.06 |
| Pro | 97.12 | 10.98 ± 0.15 | 11.23 ± 0.39 |
| Val | 99.13 | 5.62 ± 0.05 | 5.66 ± 0.09 |
| Thr | 101.11 | 3.64 ± 0.02 | 3.37 ± 0.05 |
| Cys | 103.14 | 0.28 ± 0.03 | 0.29 ± 0.03 |
| Leu | 113.16 | 7.41 ± 0.11 | 7.66 ± 0.14 |
| Ile | 113.16 | 3.99 ± 0.09 | 4.23 ± 0.08 |
| Asp | 114.08 | 3.62 ± 0.05 | 3.28 ± 0.10 |
| Asn | 114.10 | 3.63 ± 0.05 | 3.27 ± 0.10 |
| Glu | 128.11 | 8.91 ± 0.18 | 8.82 ± 0.26 |
| Gln | 128.13 | 8.92 ± 0.18 | 8.83 ± 0.26 |
| Lys | 129.18 | 6.84 ± 0.09 | 6.91 ± 0.06 |
| Met | 131.19 | 1.12 ± 0.02 | 1.52 ± 0.06 |
| His | 138.15 | 1.79 ± 0.02 | 1.72 ± 0.05 |
| Phe | 147.18 | 4.21 ± 0.04 | 4.25 ± 0.08 |
| Arg | 157.20 | 5.98 ± 0.09 | 6.36 ± 0.09 |
| Tyr | 163.18 | 3.14 ± 0.06 | 3.36 ± 0.06 |

A mean weighted mass MWM of a single amino acid residue in the first and the second fractions are calculated using the following Equation.

$$MWM = {\sum }_{AA} {m}_{AA} \frac{{x}_{AA}}{100} = \{ \begin{matrix}111.16 for the first composition \\ 111.78 Da for the second composition\end{matrix}$$

A number of amino acid residues (N) in the average peptide of a given molecular mass M for the first and second fractions are calculated using the following Equation.

$$N = \frac{MWM}{M} = \frac{1}{M} {\sum }_{AA} {m}_{AA} \frac{{x}_{AA}}{100}$$

At the upper and lower limits for molecular masses of peptides indicated by the chromatographic bands, the following numbers of amino acid residues per an average peptide were obtained:

### The First Fraction:

the minimum value of N at the lower limit, M=3000 Da

$$N = \frac{MWM}{M} = \frac{1}{M} {\sum }_{AA} {m}_{AA} \frac{{x}_{AA}}{100} = 26.99 ≅ 27 ;$$

the maximum value of N at the upper limit, M=35000 Da

$$N = \frac{MWM}{M} = \frac{1}{M} {\sum }_{AA} {m}_{AA} \frac{{x}_{AA}}{100} = 314.85 ≅ 315.$$

### The Second Fraction:

the minimum value of N at the lower limit, M=3000 Da

$$N = \frac{MWM}{M} = \frac{1}{M} {\sum }_{AA} {m}_{AA} \frac{{x}_{AA}}{100} = 26.84 ≅ 27 ;$$

the maximum value of N at the upper limit, M=6000 Da

$$N = \frac{MWM}{M} = \frac{1}{M} {\sum }_{AA} {m}_{AA} \frac{{x}_{AA}}{100} = 53.68 ≅ 54.$$

Based on the foregoing, the peptides contents of the first fraction and the second fraction include amino acid residues in the range of 27≦N≦315 and 27≦N≦54, respectively.

In order to estimate the number of different peptides contents of the first and second fractions each having N amino acid residues, the number of peptide contents of five amino acid residuals would be determine using the following equation:

$${\prod }_{i = 1}^{5} {n}_{i} = 378.$$

At the assumption that each i^{th} position at 6≦i≦N is occupied solely by two amino acid residues, i.e. nᵢ=2, the number of different peptides containing N residues reaches the following values for the first and the second fractions: first fraction:

$${\prod }_{i = 1}^{5} {n}_{i} \cdot {2}^{N - 5} = 378 \cdot {2}^{22} ≅ 1.59 \cdot {10}^{9}$$

at N=27 corresponding to the lower molecular mass limit;

$${\prod }_{i = 1}^{5} {n}_{i} \cdot {2}^{N - 5} = 378 \cdot {2}^{310} ≅ 7.88 \cdot {10}^{95}$$

at N=315 corresponding to the upper molecular mass limit.

### Second Fraction:

$${\prod }_{i = 1}^{5} {n}_{i} \cdot {2}^{N - 5} = 378 \cdot {2}^{22} ≅ 1.59 \cdot {10}^{9}$$

at N=27 corresponding to the lower molecular mass limit;

$${\prod }_{i = 1}^{5} {n}_{i} \cdot {2}^{N - 5} = 378 \cdot {2}^{49} ≅ 2.13 \cdot {10}^{17}$$

at N=54 corresponding to the upper molecular mass limit.

Based on the foregoing, the numbers of peptides in the first fraction and the second fraction, are equal to least 378 at nᵢ=1 for 6≦i≦N, and at nᵢ=2 for 6≦i≦N to the intervals [1.59·10⁹, 7.88·10⁹⁵] and [1.59·10⁹, 2.13·10¹⁷] or, respectively. Accordingly, further peptide sequencing may not be required.

(v) The 1^{st}- and 2^{nd}-Order Structures of Peptide Compositions

On the basis of SDS PAGE analysis results, indicating the occurrence of broad and structure less [˜3000, ˜35000] Da and [˜3000, ˜6000] Da bands for the first and second fractions, respectively, the primary and secondary structures of these peptide compositions was gained based on theoretical considerations of both the amino acid contents of these compositions (discussed hereinbelow), and the results of partial sequence analysis (discussed hereinabove).

Using the relative content of amino acids x_{AA} in the first and second fractions, and the number of amino acid residues,

$$N = \frac{1}{M} {\sum }_{AA} {m}_{AA} \frac{{x}_{AA}}{100}$$

of a single peptide having a molecular mass M (where, m_{AA} is the molecular mass of the amino acid residue), the number of the amino acid residues in a single peptide,

$${n}_{AA} = \frac{{x}_{AA}}{100} N ,$$

the standard deviation SD(n_{AA}) of may be calculated using the following the formula

$$SD ( {n}_{AA} ) = \frac{1}{100} \sqrt{{N}^{2} \cdot {SD}^{2} ( {x}_{AA} ) + {x}_{AA}^{2} \cdot {SD}^{2} ( N )}$$

$$where , \\ SD ( N ) = \frac{1}{100 M} \sqrt{{\sum }_{AA} {x}_{AA}^{2} {SD}^{2} ( {m}_{AA} ) + {\sum }_{AA} {m}_{AA}^{2} {SD}^{2} ( {x}_{AA} )} .$$

At the lower limit of M=3000 Da for peptide molecular masses in the second fraction, the number of amino acid residues in a single peptide is determined to be equal to,

$$N = \frac{1}{M} {\sum }_{AA} {m}_{AA} \frac{{x}_{AA}}{100} ≅ 27.$$

Therefore, a 27-amino acid peptide was selected for further analysis of the second fraction. Since N=54 corresponds to the upper limit of M=6000 Da for molecular masses of peptides in the second fraction, other peptides that may be selected for further considerations should have the N value in the following range 27≦N≦54.

Accordingly, a 40-amino acid peptide was selected for further analysis of the second fraction. Analogously, a 60-amino acid peptide was selected to represent sample of the first fraction which meets the requirement of N value being 27≦N≦315. At those N values and the x_{AA} values shown in Table 5, the numbers of amino acid residues, n_{AA}, in a single peptide of a given length were calculated, and results of such calculations are shown in the following Table.

TABLE 5
The numbers of amino acid residues in a single peptide of a given length in
dependence on the relative contents of amino acids in the the second fraction
and the first fraction preparations.

| | | | Number of amino | | Number of |
|---|---|---|---|---|---|
| | | | acid residues in | | amino acid |
| | Relative contents of | | the N-amino acid | | residues in the |
| | amino acids | | peptide in the | | N-amino acid peptide |
| | x_{AA} [%] | | second fraction | | in the first fraction |
| Amino | Second | The first | n_{AA} ± SD(n_{AA}) | | n_{AA} ± SD(n_{AA}) |
| acid AA | fraction | fraction | N = 27 | N = 40 | N = 60 |
| Gly | 9.46 ± 0.12 | 9.75 ± 0.32 | 2.55≅3 | 3.78≅4 | 5.85≅6 |
| Ala | 5.90 ± 0.06 | 6.16 ± 0.10 | 1.59≅2 | 2.36≅2 | 3.70≅4 |
| Ser | 3.87 ± 0.06 | 4.00 ± 0.01 | 1.04≅1 | 1.55≅2 | 2.40≅2 |
| Pro | 11.23 ± 0.39 | 10.98 ± 0.15 | 3.03≅3 | 4.49≅4 | 6.59≅7 |
| Val | 5.66 ± 0.09 | 5.62 ± 0.05 | 1.53≅2 | 2.26≅2 | 3.37≅3 |
| Thr | 3.37 ± 0.05 | 3.64 ± 0.02 | 0.91≅1 | 1.35≅1 | 2.18≅2 |
| Cys | 0.29 ± 0.03 | 0.28 ± 0.03 | 0.08≅0 | 0.12≅0 | 0.17≅0 |
| Leu | 7.66 ± 0.14 | 7.41 ± 0.11 | 2.07≅2 | 3.06≅3 | 4.45≅4 |
| Ile | 4.23 ± 0.08 | 3.99 ± 0.09 | 1.14≅1 | 1.69≅2 | 2.39≅2 |
| Asp | 3.28 ± 0.10 | 3.62 ± 0.05 | 0.89≅1 | 1.31≅1 | 2.17≅2 |
| Asn | 3.27 ± 0.10 | 3.63 ± 0.05 | 0.89≅1 | 1.31≅1 | 2.18≅2 |
| Glu | 8.82 ± 0.26 | 8.91 ± 0.18 | 2.38≅2 | 3.53≅4 | 5.35≅5 |
| Gln | 8.83 ± 0.26 | 8.92 ± 0.18 | 2.38≅2 | 3.53≅4 | 5.35≅5 |
| Lys | 6.91 ± 0.06 | 6.84 ± 0.09 | 1.78≅2 | 2.76≅3 | 4.10≅4 |
| Met | 1.52 ± 0.06 | 1.12 ± 0.02 | 0.41≅0 | 0.61≅1 | 0.67≅1 |
| His | 1.72 ± 0.05 | 1.79 ± 0.02 | 0.46≅0 | 0.69≅1 | 1.07≅1 |
| Phe | 4.25 ± 0.08 | 4.21 ± 0.04 | 1.15≅1 | 1.71≅2 | 2.53≅3 |
| Arg | 6.36 ± 0.09 | 5.98 ± 0.09 | 1.72≅2 | 2.54≅3 | 3.59≅4 |
| Tyr | 3.36 ± 0.06 | 3.14 ± 0.06 | 0.91≅1 | 1.34≅1 | 1.88≅2 |

Since the numbers of amino acids in a peptide composition can only be expressed in whole and positive number, the n_{AA} values are rounded, which resulted in the 27-, 41- and 59-amino acid peptides. The SD(n_{AA}) values do not exceed 3.7% of the n_{AA} values, thus their contributions to the n_{AA}, values may be neglected.

A sequence of the first five amino acid residues was determined by the means of sequence analysis. Accordingly, the remaining residues, i.e. those at the positions 6 to N, may be ordered randomly although their n_{AA}, values may be fixed for a given peptide.

An example of a sequence listing of a peptide composition consisting of 27 residues is shown as Peptide 1. A few more distinct 27-amino acid peptides (Peptides 2-4), which are equally allowable, were built as mutations of Peptide 1. These mutation-produced peptides were chosen to show the effect of terminal amino acids on these physicochemical properties of peptide compositions which determine their biological activity.

Using a similar procedure, a 41-amino acid peptide, namely Peptide 5, was built together with its mutants, i.e. Peptides 6-8. In turn, to describe the peptides of the first fraction, an example of 59-amino acid peptide is shown (Peptide 9) together with a few other 59-amino acid peptides (Peptides 10-12), which are equally allowable mutations of Peptide 9. Also these mutations were chosen to show the effect of terminal amino acids on essential physicochemical properties of these peptide compositions.

In solution at neutral pH, peptide compositions exist as zwitterions, with —NH₃⁺ and —COO⁻ terminal groups; thus the above-mentioned peptides were built as zwitterions using HyperChem 7.5. Once the primary structure of a given peptide composition had been chosen, a secondary structure of each amino acid was selected (L-isomers with torsional angles of 180 degrees set for a beta-pleated sheet with a transpeptide bond of 180 degrees) and a secondary structure of the entire peptide was determined using a molecular mechanics method AMBER 3 that was developed for protein and nucleic acid computations (Cornell et al. 1995).

The 2^{nd}-order structure of each peptide was determined by a geometry optimization, resulting in the structure that has a potential energy minimum, through the Polak-Ribiere method (at the in vacuo condition, RMS gradient of 0.05 kcal·∈⁻³·mol⁻¹, constant dielectric, scale factor of 1 for the dielectric permittivity, and van der Waals and electrostatic interaction scale factors of 0.5).

### Peptides Compositions Having 27 Amino Acid Residues

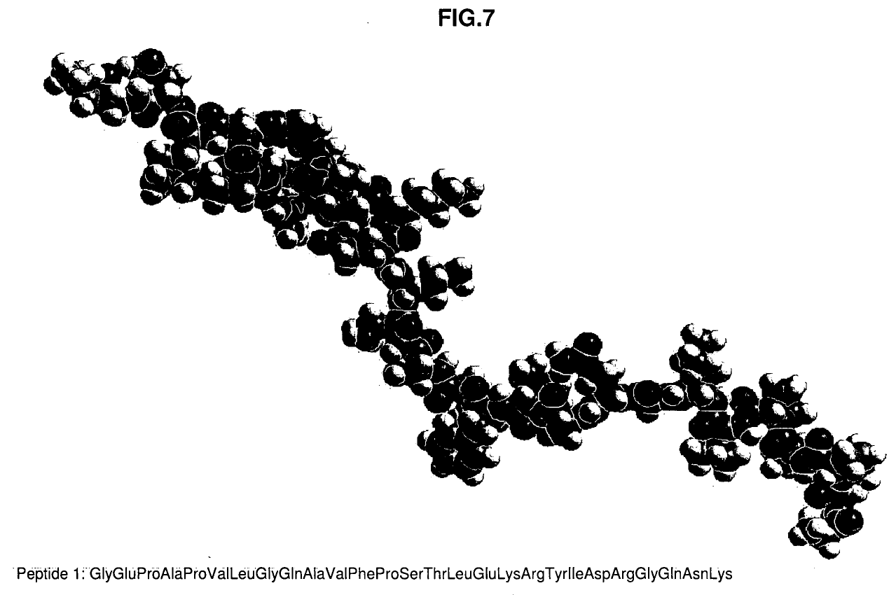

Peptide 1 was built from 27 amino acid residues (having the form of L-isomers) beginning from the N-terminus (the position 1) and ending at the C-terminus (the position 27), as shown in FIG. 7, and by the following sequence listing.

Peptide 1:
GlyGluProAlaProValLeuGlyGlnAlaValPheProSerThrLeu
Glu LysArgTyrIleAspArgGlyGlnAsnLys

Lysine that occurs in the position 27 is positively charged in order to affect significantly physicochemical properties of the peptide molecule. Accordingly; lysine was replaced by glutamic acid that is negatively charged, i.e. the following 27Lys→27Glu mutation was made in Peptide 1.

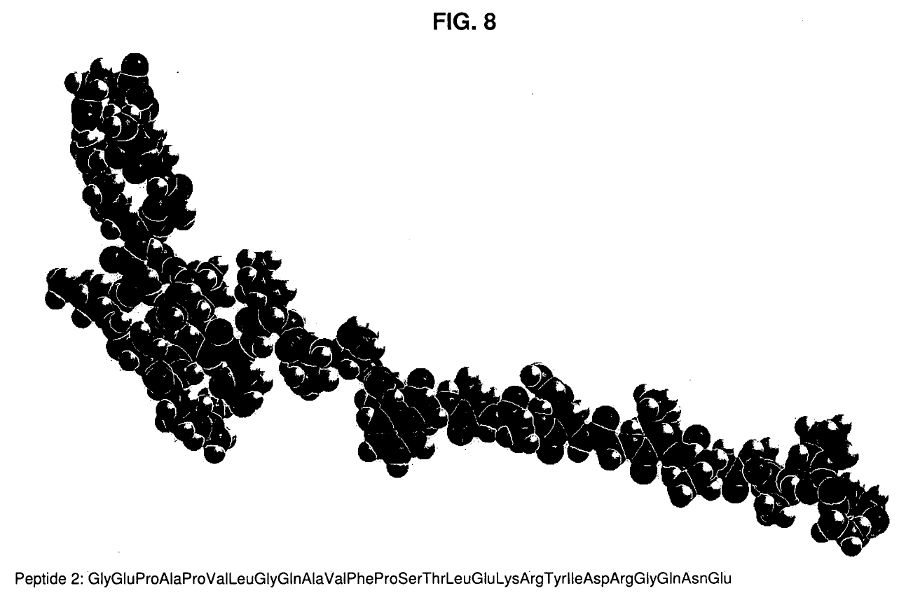

This mutation of the peptide 1 resulted in Peptide 2, as shown in FIG. 8, having a sequence listing as follows.

Peptide 2:
GlyGluProAlaProValLeuGlyGlnAlaValPheProSerThrLeu
GluLysArglyrIleAspArgGlyGlnAsnGlu

The peptide 2 has a significantly higher dipole moment and an increased lipophilicity (increased log P) in comparison to Peptide 1 (see the following Table 6).

TABLE 6
Physicochemical characteristics of selected peptides whose occurrence in the first and
second fractions is statistically allowed

| | | | | Logarithm | | | |
|---|---|---|---|---|---|---|---|
| | Number | | Hydration | of the | | | |
| | of amino | Molecular | energy | partition | Dipole | | |
| Peptide | acid | mass M | E_{H} | coefficient | moment | Refractivity^{a)} | Polarizability^{b)} |
| No. | residues N | [Da] | [kcal/mol] | logP | [D] | [Å³] | [Å³] |
| 1 | 27 | 2972.38 | −74 | 0.67 | 270 | 727 | 293 |
| 2 | 27 | 2971.30 | −68 | 5.20 | 495 | 719 | 291 |
| 3 | 27 | 2972.38 | −63 | 0.67 | 252 | 727 | 293 |
| 4 | 27 | 2971.30 | −54 | 5.20 | 98 | 719 | 291 |
| 5 | 41 | 4655.33 | −104 | −0.37 | 718 | 1143 | 461 |
| 6 | 41 | 4671.35 | −98 | −2.24 | 135 | 1145 | 462 |
| 7 | 41 | 4670.28 | −111 | 2.30 | 1562 | 1137 | 460 |
| 8 | 41 | 4656.25 | −108 | 1.76 | 1806 | 1133 | 458 |
| 9 | 59 | 6612.58 | −164 | −1.29 | 779 | 1629 | 656 |
| 10 | 59 | 6611.50 | −162 | 3.24 | 1755 | 1621 | 654 |
| 11 | 59 | 6597.50 | −170 | −2.10 | 768 | 1621 | 653 |
| 12 | 59 | 6611.52 | −169 | −1.56 | 766 | 1626 | 655 |

^{a)}Calculated according to Ghose and Crippen 1987, and Viswanadhan et al. 1989.
^{b)}Calculated as polarizability volume according to Miller 1990.

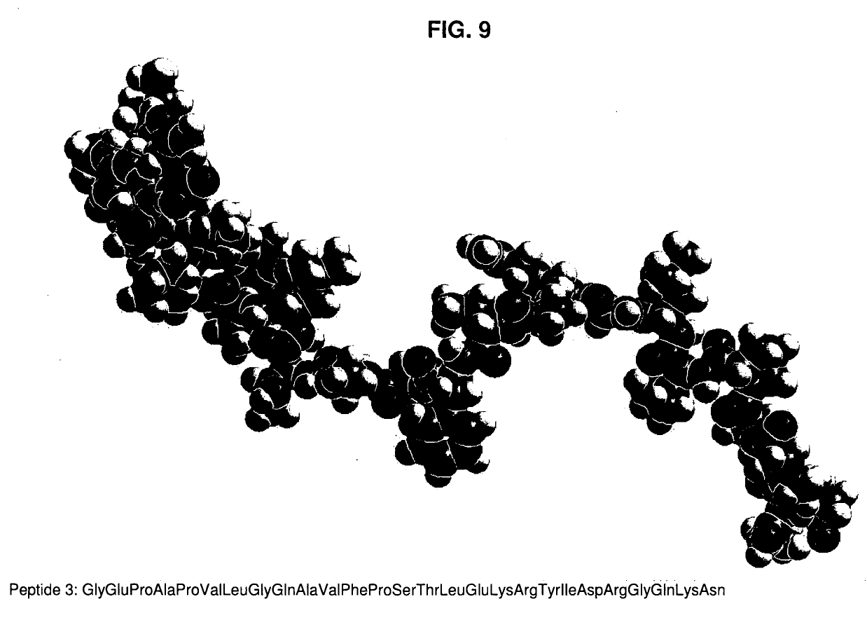

To demonstrate the effect produced by the presence of a neutral but polar amino acid in the position 27, the 26Asn→26Lys and 27Lys→27Asn mutations were made in Peptide 1, leading to formation of Peptide 3, as shown in FIG. 9.

Peptide 3:
GlyGluProAlaProValLeuGlyGlnAlaValPheProSerThrLeu
GluLysArgTyrIleAspArgGlyGlnLysAsn

In result, these two mutations caused a decrease in the dipole moment of the molecule and a decrease in the released (because of E_{H}<0) energy of hydration at the unchanged hydrophilicity, as seen from the Table 6.

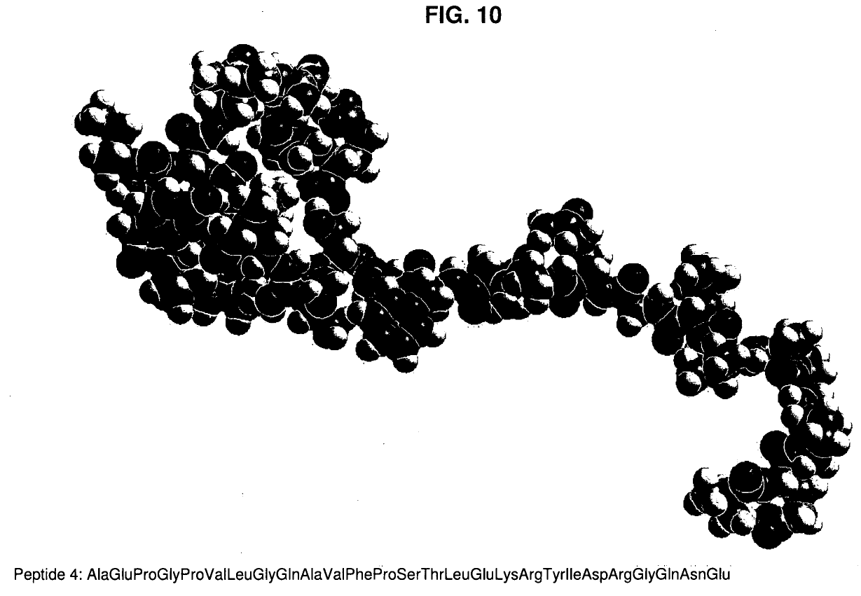

The 1Gly→1Ala and 4Ala→4Gly mutations in Peptide 2, in which neutral and polar glycine is replaced by neutral and nonpolar alanine in the position 1 and vice versa in the position 4, leading to Peptide 4, as shown in FIG. 10, does not changes high lipophilicity of the peptide. However, such mutation significantly diminishes dipole moment and the absolute value of the hydration energy of the peptide 4 as shown in Table 6.

Peptide 4:
AlaGluProGlyProValLeuGlyGlnAlaValPheProSerThrLeu
GluLysArgTyrIleAspArgGlyGlnAsnGlu

All of the above-mentioned peptides are statistically allowed in the molecular weight interval [˜3000, ˜6000] Da, which is a typical molecular weight for the second fraction.

### Peptides Consisting of 41 Amino Acid Residues

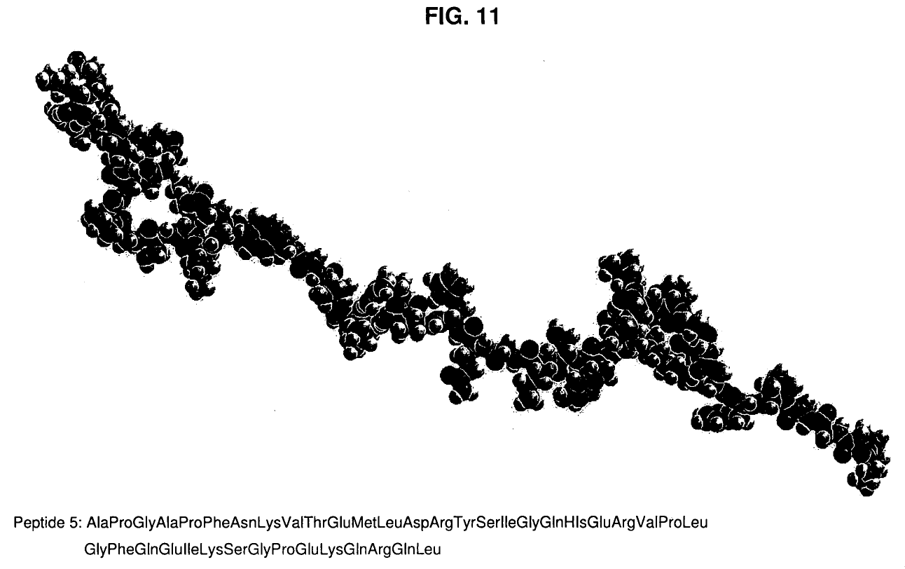

Peptide 5, as shown in FIG. 11, was built from 41 amino acid residues (having the form of L-isomers) beginning from the N-terminus (the position 1) and ending at the C-terminus (the position 41):

Peptide 5:
AlaProGlyAlaProPheAsnLysValThrGluMetLeuAspArgTyr
SerIleGlyGlnHisGluArgValProLeuGlyPheGlnGluIleLys
SerGlyProGluLysGlnArgGlnLeu

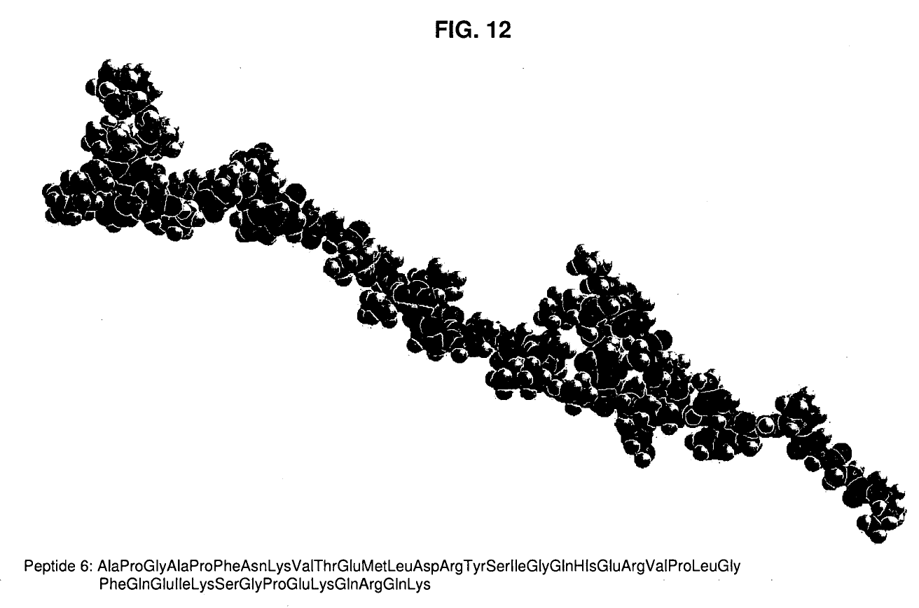

The 41Leu→41Lys mutation in Peptide 5, in which a neutral and nonpolar amino acid was replaced by a positively charged one, resulted in Peptide 6, as shown in FIG. 12, whose dipole moment and log P significantly dropped (see Table 6). Changes in the other parameters like the hydration energy, refractivity or polarizability were very small (see Table 6).

Peptide 6:
AlaProGlyAlaProPheAsnLysValThrGluMetLeuAspArgTyr
SerIleGlyGlnHisGluArgValProLeuGlyPheGlnGluIleLys
SerGlyProGluLysGlnArgGlnLys

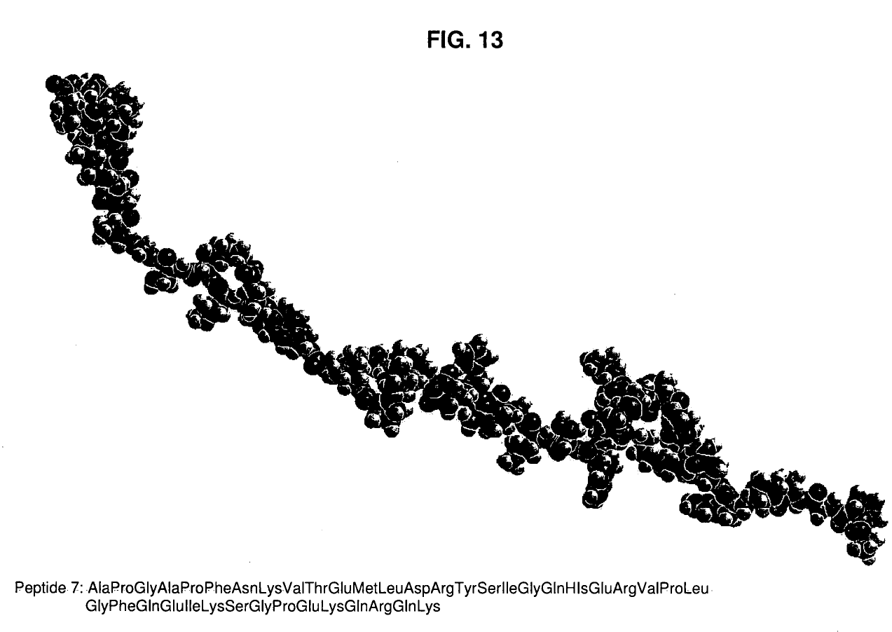

The 41Leu→41Glu mutation in Peptide 5, in which a neutral and nonpolar amino acid was replaced by a negatively charged one, resulted in Peptide 7, as shown in FIG. 13, whose dipole moment and log P significantly increased (see Table 6).

Peptide 7:
AlaProGlyAlaProPheAsnLysValThrGluMetLeuAspArgTyr
SerIleGlyGlnHisGluArgValProLeuGlyPheGlnGluIleLys
SerGlyProGluLysGlnArgGlnGlu

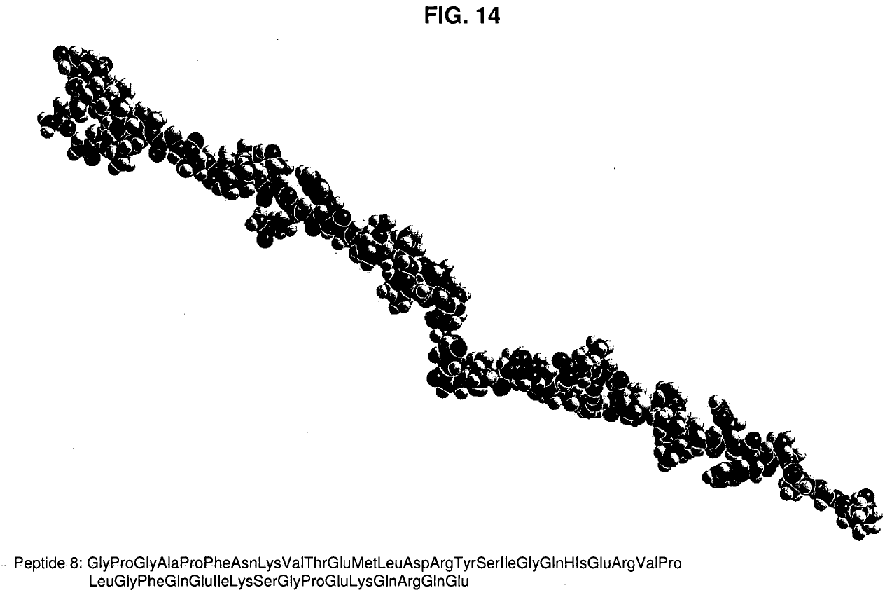

The 1Ala→1Gly and 41Leu→41Glu mutations in Peptide 5, in which neutral and nonpolar terminal amino acids were replaced by a neutral and polar or negatively charged amino acid, respectively, resulted in Peptide 8, as shown in FIG. 14, whose dipole moment and log P significantly increased (Table 6).

Peptide 8:
GlyProGlyAlaProPheAsnLysValThrGluMetLeuAspArgTyr
SerIleGlyGlnHisGluArgValProLeuGlyPheGlnGluIleLys
SerGlyProGluLysGlnArgGlnGlu

### Peptides Consisting of 59 Amino Acid Residues

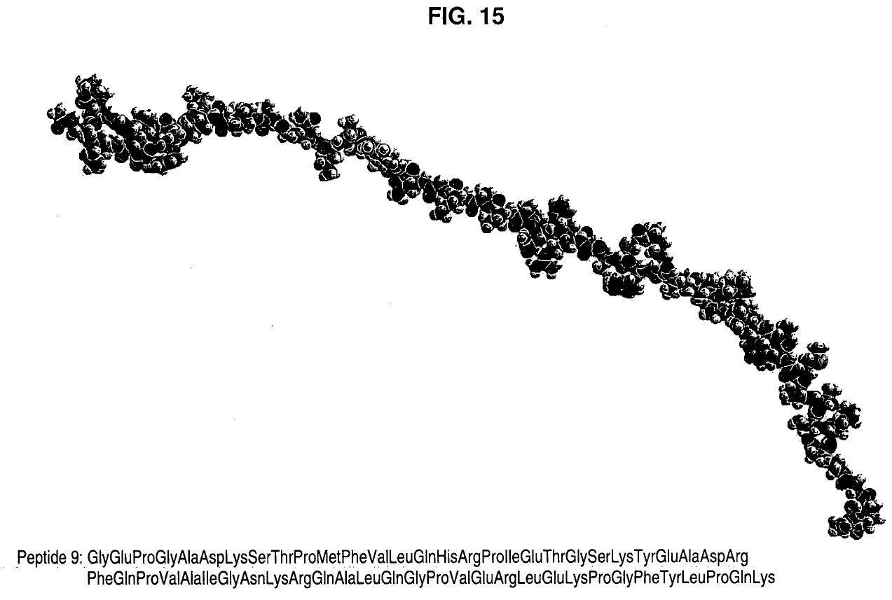

Peptide 9, as shown in FIG. 15, was built from 59.

Peptide 9:
GlyGluProGlyAlaAspLysSerThrProMetPheValLeuGlnHis
ArgProIleGluThrGlySerLysTyrGluAlaAspArgPheGlnPro
ValAlaIleGlyAsnLysArgGlnAlaLeuGlnGlyProValGluArg
LeuGluLysProGlyPheTyrLeuProGlnLys

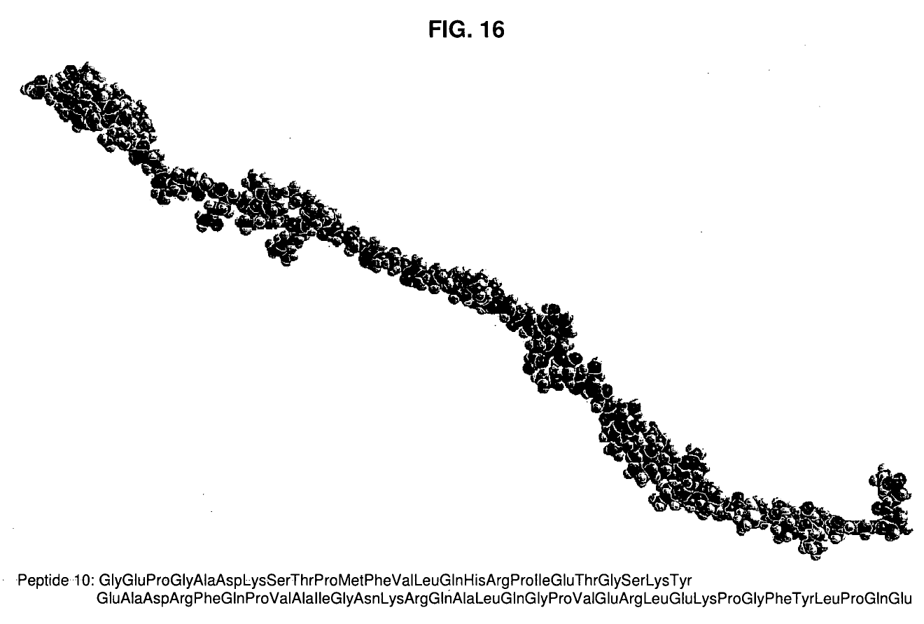

The 59Lys→59Glu mutation in Peptide 9 resulted in Peptide 10, as shown in FIG. 16, whose dipole moment dramatically increased as well as log P (see Table 6). The latter change indicates that the peptide that was hydrophilic (log P=−1.29) mutated into lipophilic one (log P=3.24).

Peptide 10:
GlyGluProGlyAlaAspLysSerThrProMetPheValLeuGlnHis
ArgProIleGluThrGlySerLysTyrGluAlaAspArgPheGlnPro
ValAlaIleGlyAsnLysArgGlnAlaLeuGlnGlyProValGluArg
LeuGluLysProGlyPheIyrLeuProGlnGlu

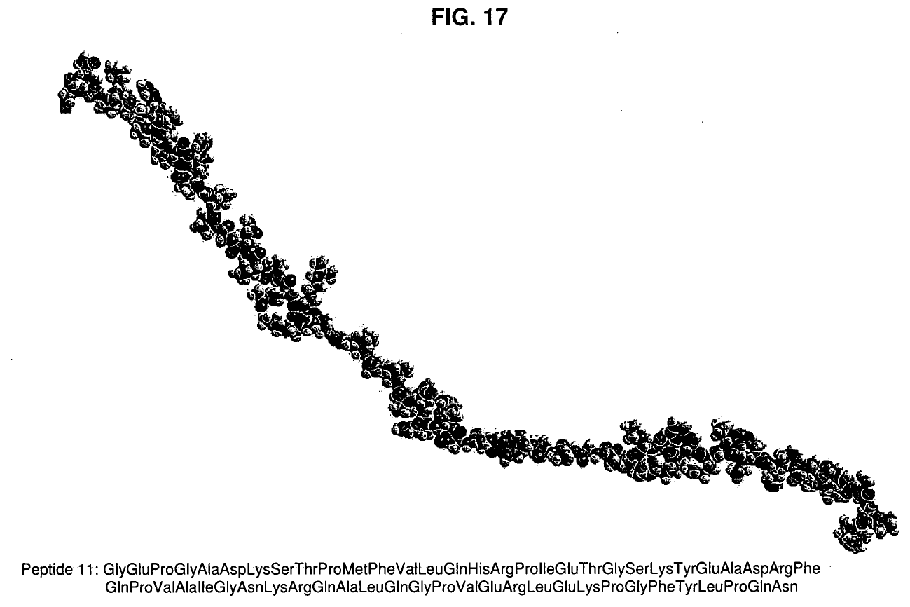

The 59Lys→59Asn mutation in Peptide 9 resulted in Peptide 11, as shown in FIG. 17, for which only log P changed significantly, from −1.29 to −2.10 (see Table 6), indicating more hydrophilic character of Peptide 11.

Peptide 11:
GlyGluProGlyAlaAspLysSerThrProMetPheValLeuGlnHis
ArgProIleGluThrGlySerLysTyrGluAlaAspArgPheGlnPro
ValAlaIleGlyAsnLysArgGlnAlaLeuGlnGlyProValGluArg
LeuGluLysProGlyPheTyrLeuProGlnAsn

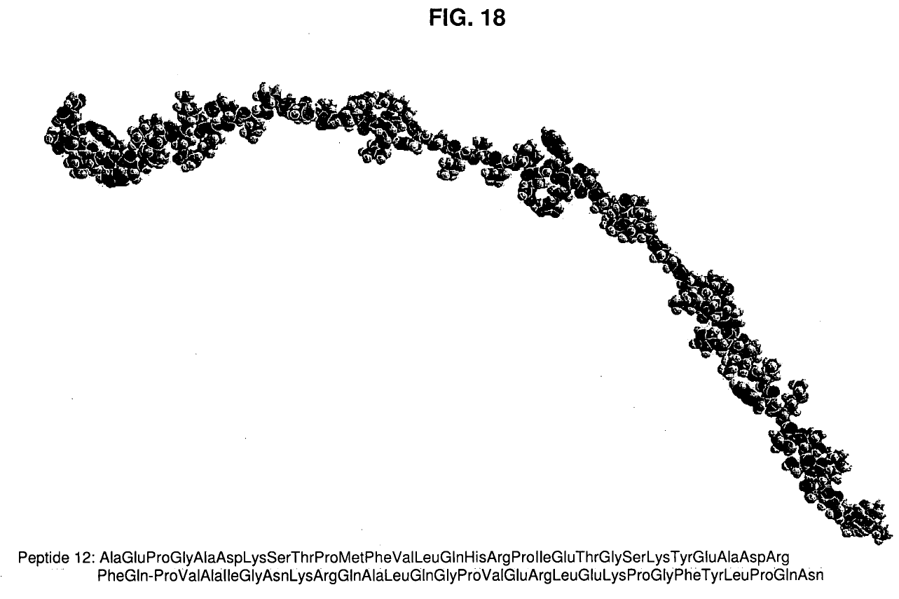

The two mutations, 1Gly→1Ala and 59Lys→59Asn, transform Peptide 9 into Peptide 12, as shown inn FIG. 18, with only slightly decreased values of the dipole moment and log P (Table 6).

Peptide 12:
AlaGluProGlyAlaAspLysSerThrProMetPheValLeuGlnHis
ArgProIleGluThrGlySerLysTyrGluAlaAspArgPheGlnPro
ValAlaIleGlyAsnLysArgGlnAlaLeuGlnGlyProValGluArg
LeuGluLysProGlyPheTyrLeuProGlnAsn

The parameters shown in Table 7 have widely been employed for the last decade or so in drug analysis to evaluate quantitatively how “druglike” a compound is (Ghose et al. 1999; Lipinski et al. 2001). The concept of druglikeness leads from the molecular structure of a given compound to its role like a drug with a given biological activity, which calls for optimal solubility of the compound in both water and lipids, because in the case of a sublingual administration, the compound has to route both inter- and intracellularly through the oral mucosa to the oral MALT.

This route needs some affinity to both aqueous and lipid phases in the oral cavity and other connected body portions. In other words, the logarithm of the octanol-water partition coefficient should be between −0.4 and 5.6. The peptides shown in Table 6 generally belong to this interval, particularly those which are more hydrophobic (log P>1) than hydrophilic (log P<1). It should be emphasized, however, that the log P criterion (like a few others, e.g. on the molecular mass lesser than 480 Da, molecular refractivity from 40 to 130 Å³, number of hydrogen bond donors/acceptors not higher than 5 or 10, respectively) has not been established on the basis of peptides pretending to the name of drugs.

Therefore, the Lipinski's rule of five serves only to provide approximate indicator of the peptide druglikeness. The high values of peptides' dipole moments point out significance of the dipole-dipole and ion-dipole interactions between the peptides (that are also zwitterions) and water molecules, although the distinct hydrophilicity of certain peptides, e.g., peptides 6, 9, 11, 12, as shown in Table 6.

The peptides discussed above (shown in FIGS. 7-18) are assumed to be stable, due to the relatively high values of the hydration energy (Table 6), serving as a factor determining the molecular stability (Ooi et al. 1987).

This stability, however, allows a significant deformability of the peptide molecules in the external electric field of surrounding dipoles of water or other molecules in the body fluids, resulting in the formation of induced dipole moments, as it is indicated by the polarizability values (Table 6). In consequence, the peptides are characterized by a high polarization that makes them very responsible to various interactions with the surrounding dipoles and charged molecules in the body compartments.

### II. Amino Acid Contents of the Peptide Compositions

Liquid-phase hydrolysis of lyophilized samples of the peptide compositions was performed in 6M HCl containing 0.5% phenol (for tyrosine protection) at 110° C. for 24 hours under argon gas atmosphere. Cysteine content was analyzed after oxidation of samples by performic acid (to obtain stable form of cysteic acid) followed by standard hydrolysis with HCl. Obtained hydrolyzates were determined by ion-exchange chromatography with post-column derivatization with ninhydrin using an automatic amino acid analyzer AAA 400 (obtained from Ingos, Czech Republic) according to standard protocol of the manufacturer.

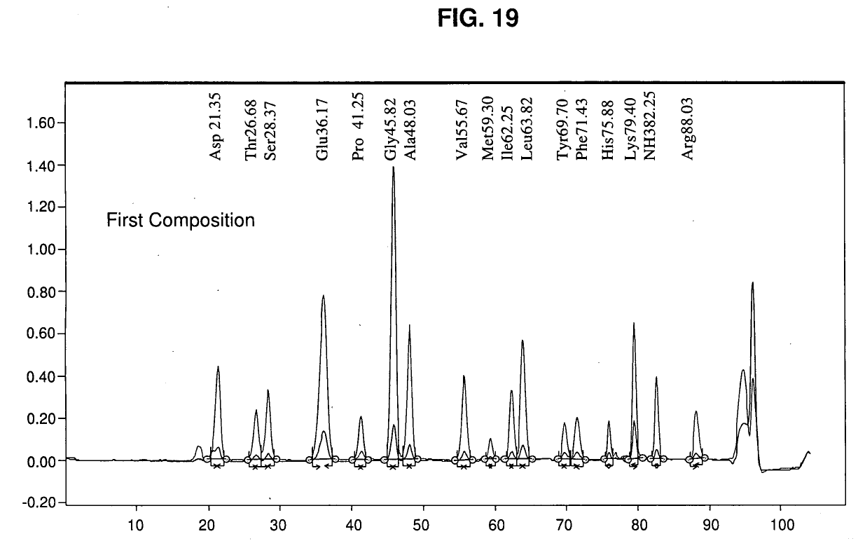

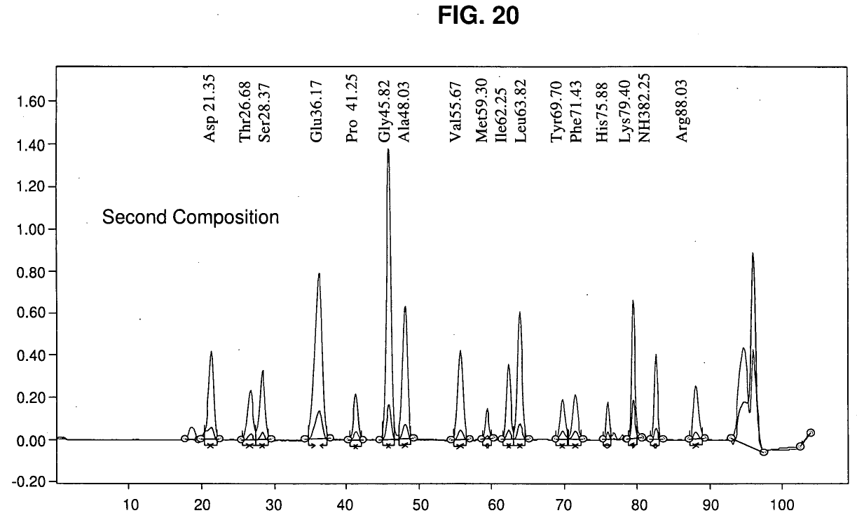

The results of the amino acid analysis, conducted according to this procedure, are disclosed in FIG. 19 and FIG. 20 for the first and second fractions, respectively. The amino acid compositions of the analyzed samples (analyzed in triplicates) are shown in the following Tables 7 and 8.

It was found that all amino acids were detected (tryptophan not included), where determined aspartic acid includes asparagine and glutamic acid includes glutamine. Tryptophan is destroyed during the measurement's conditions.

TABLE 7
The percentage amino acid content of lyophilized samples (g/100 g
sample)

| | | | | |
|---|---|---|---|---|
| 1 | 2 | 3 | Mean | SD |

First Fraction

| | | | | | |
|---|---|---|---|---|---|
| Asp | 4.7594 | 4.8141 | 4.8044 | 4.7926 | 0.02916 |
| Thr | 2.3870 | 2.4053 | 2.4059 | 2.3994 | 0.01076 |
| Ser | 2.6290 | 2.6364 | 2.6382 | 2.6345 | 0.00490 |
| Glu | 11.6114 | 11.7617 | 11.8353 | 11.7361 | 0.11412 |
| Pro | 7.1372 | 7.2347 | 7.3268 | 7.2329 | 0.09482 |
| Gly | 6.1845 | 6.5503 | 6.5318 | 6.4222 | 0.20609 |
| Ala | 3.9873 | 4.0913 | 4.1001 | 4.0596 | 0.06272 |
| Val | 3.6637 | 3.7283 | 3.7100 | 3.7007 | 0.03326 |
| Met | 0.7297 | 0.7476 | 0.7276 | 0.7350 | 0.01101 |
| Ile | 2.5661 | 2.6667 | 2.6527 | 2.6285 | 0.05451 |
| Leu | 4.8022 | 4.9348 | 4.9123 | 4.8831 | 0.07097 |
| Tyr | 2.0329 | 2.0815 | 2.0993 | 2.0712 | 0.03437 |
| Phe | 2.7710 | 2.7528 | 2.7999 | 2.7745 | 0.02377 |
| His | 1.1677 | 1.1736 | 1.1927 | 1.1780 | 0.01309 |
| Lys | 4.4444 | 4.5426 | 4.5421 | 4.5097 | 0.05658 |
| Arg | 3.9137 | 3.9056 | 4.0064 | 3.9419 | 0.05601 |
| Cys | 0.1874 | 0.2011 | 0.1621 | 0.1835 | 0.01979 |
| Σ | 65.9745 | 68.2282 | 69.4475 | 65.8834 | |

Second Fraction

| | | | | | |
|---|---|---|---|---|---|
| Asp | 4.0494 | 4.1520 | 4.1649 | 4.1221 | 0.06332 |
| Thr | 2.0906 | 2.1380 | 2.1426 | 2.1237 | 0.02881 |
| Ser | 2.4023 | 2.4374 | 2.4774 | 2.4390 | 0.03762 |
| Glu | 10.9497 | 11.1142 | 11.2734 | 11.1124 | 0.16186 |
| Pro | 6.8388 | 7.0579 | 7.3284 | 7.0750 | 0.24528 |
| Gly | 5.8899 | 5.9471 | 6.0324 | 5.9564 | 0.07174 |
| Ala | 3.6761 | 3.7272 | 3.7501 | 3.7178 | 0.03788 |
| Val | 3.5308 | 3.5342 | 3.6256 | 3.5635 | 0.05375 |
| Met | 0.9378 | 0.9612 | 0.9671 | 0.9554 | 0.01548 |
| Ile | 2.6457 | 2.6301 | 2.7201 | 2.6653 | 0.04811 |
| Leu | 4.7662 | 4.7862 | 4.9209 | 4.8244 | 0.08414 |
| Tyr | 2.0810 | 2.1111 | 2.1578 | 2.1166 | 0.03870 |
| Phe | 2.6475 | 2.6520 | 2.7288 | 2.6761 | 0.04572 |
| His | 1.0544 | 1.0828 | 1.1138 | 1.0837 | 0.02972 |
| Lys | 4.3163 | 4.3479 | 4.3915 | 4.3519 | 0.03776 |
| Arg | 3.9405 | 4.0340 | 4.0394 | 4.0046 | 0.05562 |
| Cys | 0.1710 | 0.182 | 0.2030 | 0.1853 | 0.01626 |
| Σ | 62.9877 | 64.8951 | 67.0373 | 62.9734 | |

TABLE 8
The weight percentage amino acid composition of protein (g/100 g
protein)

| | | | | |
|---|---|---|---|---|
| 1 | 2 | 3 | Mean | SD |

First Fraction

| | | | | | |
|---|---|---|---|---|---|
| Asp | 7.22 | 7.31 | 7.29 | 7.27 | 0.044 |
| Thr | 3.62 | 3.65 | 3.65 | 3.64 | 0.016 |
| Ser | 3.99 | 4.00 | 4.00 | 4.00 | 0.007 |
| Glu | 17.62 | 17.85 | 17.96 | 17.81 | 0.173 |
| Pro | 10.83 | 10.98 | 11.12 | 10.98 | 0.144 |
| Gly | 9.39 | 9.94 | 9.91 | 9.75 | 0.313 |
| Ala | 6.05 | 6.21 | 6.22 | 6.16 | 0.095 |
| Val | 5.56 | 5.66 | 5.63 | 5.62 | 0.050 |
| Met | 1.11 | 1.13 | 1.10 | 1.12 | 0.017 |
| Ile | 3.89 | 4.05 | 4.03 | 3.99 | 0.083 |
| Leu | 7.29 | 7.49 | 7.46 | 7.41 | 0.108 |
| Tyr | 3.09 | 3.16 | 3.19 | 3.14 | 0.052 |
| Phe | 4.21 | 4.18 | 4.25 | 4.21 | 0.036 |
| His | 1.77 | 1.78 | 1.81 | 1.79 | 0.020 |
| Lys | 6.75 | 6.89 | 6.89 | 6.84 | 0.086 |
| Arg | 5.94 | 5.93 | 6.08 | 5.98 | 0.085 |
| Cys | 0.28 | 0.31 | 0.25 | 0.28 | 0.030 |
| Σ | 98.62 | 100.52 | 100.86 | 100.00 | |

Second Fraction

| | | | | | |
|---|---|---|---|---|---|
| Asp | 6.43 | 6.59 | 6.61 | 6.55 | 0.101 |
| Thr | 3.32 | 3.40 | 3.40 | 3.37 | 0.046 |
| Ser | 3.81 | 3.87 | 3.93 | 3.87 | 0.060 |
| Glu | 17.39 | 17.65 | 17.90 | 17.65 | 0.257 |
| Pro | 10.86 | 11.21 | 11.64 | 11.23 | 0.389 |
| Gly | 9.35 | 9.44 | 9.58 | 9.46 | 0.114 |
| Ala | 5.84 | 5.92 | 5.96 | 5.90 | 0.060 |
| Val | 5.61 | 5.61 | 5.76 | 5.66 | 0.085 |
| Met | 1.49 | 1.53 | 1.54 | 1.52 | 0.025 |
| Ile | 4.20 | 4.18 | 4.32 | 4.23 | 0.076 |
| Leu | 7.57 | 7.60 | 7.81 | 7.66 | 0.134 |
| Tyr | 3.30 | 3.35 | 3.43 | 3.36 | 0.061 |
| Phe | 4.20 | 4.21 | 4.33 | 4.25 | 0.073 |
| His | 1.67 | 1.72 | 1.77 | 1.72 | 0.047 |
| Lys | 6.85 | 6.90 | 6.97 | 6.91 | 0.060 |
| Arg | 6.26 | 6.41 | 6.41 | 6.36 | 0.088 |
| Cys | 0.27 | 0.29 | 0.32 | 0.29 | 0.026 |
| Σ | 98.43 | 99.88 | 101.69 | 100.00 | |

### Part 2: Physical/Chemical and Biological Functions of the Peptide Compositions Through In Vitro Experiments

### I. Free Radical Scavenging Functions of the Peptide Compositions

In order to evaluate free radical scavenging functions of the peptide compositions, experimentally obtained spectra were analyzed using Eleana 0.8.3 program (Sarewicz 2006). The spectra of the trapped radicals were simulated using WinSIM (Duling 1994). The amounts of generated radicals were determined by comparison double integrals of the spin adduct with TEMPOL standard (Barr et al. 2003).

Further, in order to evaluate the ability of the first and second fractions to scavenge the hydroxyl radical the intensity of 5-tert-butoxycarbonyl 5-methyl-1-pyrroline N-oxide (BMPO) adduct was measured in the presence and absence of the investigated substances.

It was found that in the presence of the first and second fractions, all kinds of radicals are somewhat consumed. Products of decomposition of cumene hydroperoxide are essentially completely scavenged, whereas the other types of radicals are eliminated less. The effectiveness of the first and second fractions decreases in the order: hydroxyl radical>azide radical>superoxide radical>carbon-centered radical. It is also concluded that carbon centered radicals are somewhat resistant to scavenging by the second fraction and the first fraction.

### II. Antioxidative Function of the Second Fraction According to a Thiobarbituric Acid (TBA) Test Using a Human Erythrocyte Model

Oxidation stress has been commonly recognized in the last decades as one of causative factors that induce oxidative injury and perturb cellular functions in the human body. Non-scavenged free radicals, mainly oxygen radicals, initiate a variety of chain reactions, in which various cellular components, from cytoplasmic structures to cell membrane, undergo oxidative damage, resulting in dysfunction or even death of cells.

In the present study, an erythrocyte model for oxidation stress was employed to evaluate antioxidant effects produced by the second fraction. The oxidation of isolated, whole human erythrocytes was performed using cumene hydroperoxide (CumOOH) under the condition of strong oxidative stress (resulting from the 0.815 mmol/l CumOOH concentration used in this study in comparison to the 0.2 mmol/l value attributed to the condition of strong oxidation; Onaran et al. 1997), in which antioxidant defense mechanisms in the cells were overwhelmed and cell lysis is allowed.

The antioxidative activity of the second fraction was determined using isolated human erythrocytes that were oxidized CumOOH under strong oxidative conditions allowing membrane degradation through oxidation of membrane polyunsaturated fatty acids, hemoglobin oxidation, and cell lysis. Using the TBA assay in its single measurement spectrophotometric version, a dose dependent reduction in the total amount of TBA-reactive species, being produced by CumOOH in the erythrocytes, was discovered and modeled as an exponentially decreasing function of the second fraction.

It was found that the level of free radicals and their products such as superoxide anion radical, hydrogen peroxide, which can also determine, to some extent, nonenzymic lipid oxidation, may be controlled enzymatically using the second fraction.

### III. Enhancement of Enzymatic Activity of Superoxide Dismutase (Sod) in Human Blood and of Glutathione Peroxidase (Gpx) in Human Erythrocytes by the Second Fraction

In a study conducted with the human blood, the second fraction, at a 16.5% (v/v) concentration, was found to a) restore entirely activity of superoxide dismutase (SOD) in isolated human red blood cells, previously inhibited by CumOOH-derived free radicals, and b) enhance activity of SOD by 11.0 percents in native red blood cells. Also, the second fraction was found to a) restore in 73.4 percent activity of glutathione peroxidase (GPx) in whole blood, inhibited by CumOOH-derived free radicals, and b) enhance activity of GPx by 22.3 percent in CumOOH— untreated whole blood.

### IV. Antimalarial Function of the First Fraction Resulting From its Prooxidative Activity in Human Erythrocytes Revealed by Chemiluminescence Measurements

In a study conducted, it was found that the first fraction produces significant prooxidant effects in all subcellular components of the erythrocyte, which are essential for protection against the erythrocyte-stage malaria parasites.

### V. Effect of First and Second Fractions on In Vitro Generated Free Radicals Measured by Spin Trapping Electron Paramagnetic Resonance Spectroscopy

It is know that electron Paramagnetic Resonance Spin Trapping (EPR) technique provides the most direct means of investigation of free radicals. The electron paramagnetic resonance spectroscopy (EPR), also known as electron spin resonance (ESR) is, by far, the most suitable method for detecting, quantifying and identification of the substances that posses at least one unpaired electron spin (Vergely et al. 2003). Such compounds that give the EPR spectrum include transition metals (like Fe, Cu, Ni) and free radicals.

The later group encompasses the dangerous organic reactants that can be generated inside the living organisms as the effect of side reactions catalyzed by many oxidoreductases (Raha and Robinson 2000; Muller et al. 2002). The residual production of free radicals is extremely dangerous due to their high reactivity and tendency to trigger chain reactions, leading to membranes and macromolecular damage (Dean et al. 1997; Dikalov and Mason 2001).

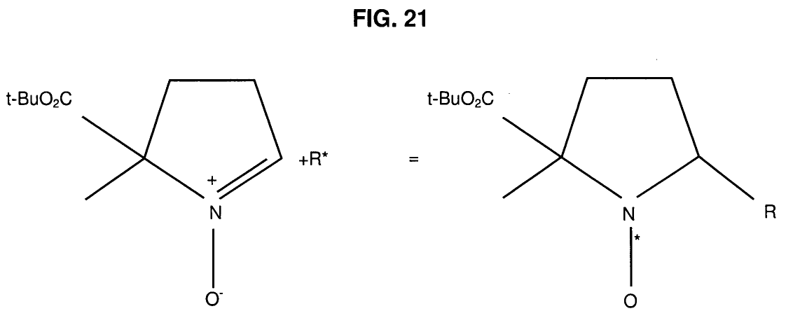

By virtue of the very short lifetime of the free radicals it is not possible to directly detect them under steady-state conditions by EPR. Therefore the spin trapping technique is commonly used to both detection and identification of the generated radical (Zhao et al. 2001, Khan et al. 2003). Principle of this technique is briefly summarized in FIG. 21.

As shown in FIG. 21, the diamagnetic (therefore, EPR silent) nitrone spin trap (BMPO in this case) reacts with a volatile radical R yielding formation of more stable and paramagnetic compound—the nitroxide adduct, whose lifetime is usually several orders of magnitude longer than trapped radical (Keszler et al. 2003). What is more, the shape of EPR spectrum depends on the type of the trapped radical. This provides the convenient means of identification of the radicals (Dikalov and Mason 2001, Vergely et al. 2003).

Possibility of identification is the consequence of the different dipolar coupling of unpaired electron spin with nuclear magnetic momentum of surrounded nuclei (usually hydrogen and nitrogen). The extend of this coupling is defined as hyperfine (or super hyperfine) splitting constant a in units of magnetic induction (T or G). Different radicals often set the unique values of spiting constants giving distinct shape of EPR spectrum when trapped by nitrone spin trap.

Given that, spin labeling EPR spectroscopy is generally very attractive method of study the antioxidant properties of variety of compounds. In this work, this technique was utilized for survey of the effect of the peptide preparations: first and second fractions on the in vitro generated radicals. The considered radicals include hydroxyl radical, carbon centered, nitrogen centered (azide), cumene hydroperoxide derived radicals and finally, the superoxide radical. From biological point of view, superoxide anion is one of the most important radical.

### Materials and Methods:

### Materials:

Ferrous ammonium sulphate (Mohr's salt) were from Reachim. Sodium azide, hydrogen peroxide, sodium phosphate and ethanol were obtained from POCH. Dimethyl sulfoxide (DMSO), xanthine, superoxide dismutase from bovine erythrocytes and deferoxamine were purchased from Sigma-Aldrich. Xanthine oxidase from buttermilk was obtained from Fluka. 1-hydroxy-3-carboxy-pyrrolidine (CPH) was from Alexis Biochemicals. 5-tert-butoxycarbonyl 5-methyl-1-pyrroline N-oxide (BMPO) was synthesized by Dr. Joy Joseph at Dept. of Biophysics, Medical College of Wisconsin, USA. Cumene hydroperoxide was kindly supplied by Dr. hab. Bonawentura Kochel. Distilled water was additionally purified using Millipore MiliQ Purification System to obtain the resistance up to 18.2 MΩ. All reagents used were of analytical grade.

Mohr's salt solution was prepared daily by dissolving the solid in thoroughly chelexed water containing 3·10⁻⁵ M of H₂SO₄. Before addition of solid salt, the acidified water was bubbled with high purity argon gas, for at least one hour. In order to minimize the oxidation of the Fe^{II} ions, the argonated solution of Mohr's salt was stored in sealed container at 0° C., throughout the given experimental series. Such prepared iron salt was stable for at least several hours giving reproducible effects on the concentration of generated radicals.

Both of the peptide preparations were manufactured by BioCentrum Ltd. according to the Batch History Record that had been obtained from Immune@Work Inc. Concentration of the dry mass was obtained by water evaporation from the samples of first and second fractions on speed vacuum centrifuge overnight. Total mass was determined by weighting the solid on the analytical second fraction.

### Methods:

Experimentally obtained spectra were analyzed using Eleana 0.8.3 program (Sarewicz 2006). The spectra of the trapped radicals were simulated using WinSIM developed by Dr. Duling from the laboratory of Molecular Biophysics, NIEHS (Duling 1994). The amount of generated radicals were determined by comparison double integrals of the spin adduct with TEMPOL standard (Barr et al. 2003).

### Generation and Measurement of Hydroxyl Radical:

The most reactive hydroxyl radical (*OH) was chemically generated in the well-known Fenton reaction (Barr 2003) in the presence of BMPO, according to the following scheme:

H₂O₂+Fe²⁺---->HO⁻+Fe³⁺+HO*(k≅60 M⁻¹s⁻¹, Bartosz 2003)*OH+BMPO-->BMPO*-OH (k∝10⁴ M⁻¹s⁻¹, Bartosz 2003)

To evaluate the ability of the peptide preparations having first and second fractions to scavenge the hydroxyl radical the intensity of BMPO adduct was measured in the presence and absence of the investigated substances. The control sample (without first fraction/second fraction) contained 250 mM of hydrogen peroxide, and 40 mM of BMPO in water. The use of any buffering agents was avoided because they may induce precipitation (in the presence of phosphates) or binding (Tris) of iron ions (Barr 2003; Bartosz 2003). The samples of interest contained the same quantities of the corresponding reactants supplemented with first and second fractions in the final concentration of 26 mg/ml.

The reaction was started by injection of Mohr's salt into the sample to get its final concentration 250 μM. Immediately after starting the reaction about 35 μl of the sample was transferred into the EPR tube followed by inserting the tube into the resonator. The time interval between the injection of the Mohr's salt and the start of the spectrum acquisition was 80 s and did not change throughout the given experimental series.

### Generation and Measurement of Carbon-Centered Radical:

The carbon-centered radical was generated by triggering the Fenton reaction in the presence of 24% of ethanol. Due to rapid reaction of the hydroxyl radical with ethanol (reaction constant in the order of 2·10⁹ M⁻¹s⁻¹) nearly the whole quantity of hydroxyl is converted into carbon centered radical, followed by reaction of the radical with BMPO spin trap, according to the following scheme:

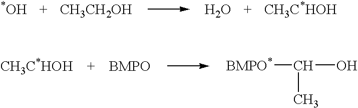

The measurements of the forming radical as well as the peptide preparations were performed in the same way as in the case of hydroxyl radical (see Generation and measurement of hydroxyl radical subsection) using the same concentrations of reactants.

### Generation of Azide Radical:

Similarly to the carbon-centered radical, the azide radical (*N₃) was generated in Fenton reaction in the presence of 250 mM of sodium azide. The reaction rate constant of N₃⁻ with *OH is even faster than with ethanol by approx. one order of magnitude. Thus in the condition used, the majority of generated *OH is consumed by azide anion producing nitrogen centered radical, further trapped by BMPO:

*OH+N₃⁻+H⁺-->H₂O+*N₃ (k in the order of 10¹⁰ M⁻¹s⁻¹)BMPO+*N₃--->BMPO*-N₃

Again, the effect of first and second fractions was evaluated in the same way, as in the case of hydroxyl radical (see Generation and measurement of hydroxyl radical).

### Generation of Cumene Hydroperoxide Derived Radicals:

Radicals generated by the decomposition of cumene hydroperoxide were formed by two methods. First, the radical was generated by injection of Mohr's salt (final concentration 100 μM) into the sample containing 40 mM BMPO and 0.08% of the cumene hydroperoxide. Second, spontaneously generated radical that comes from decomposition of the hydroperoxides catalyzed by trace amount of transition metals (Bartosz 2003) was measured.

In this case, the sample contained 8% of cumene hydroperoxide, 50% of DMSO and 40 mM BMPO in 25 mM phosphate buffer, pH 7.4. The effect of first and second fractions was measured by comparison of amplitude of the BMPO adduct signal in the presence and absence of investigated peptides supplemented in 26 mg/ml.

### Generation Superoxide Radical:

Superoxide radical was generated by the well-known Xanthine (X)/Xanthine Oxidase (XO) system (Zhao et al. 2001; Bartosz 2003; Keszler et al. 2003). In this reaction, in the presence of O₂, xanthine is oxidized by XO to uric acid (UA), concomitantly producing some quantities of superoxide anion radical; according to the following scheme:

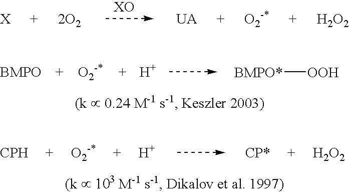

By virtue of very slow reaction of superoxide radical with BMPO (and all nitrone spin traps), it was only used to confirm the generation of this radical without any derivatives. The measurement of the sample contained 500 mM of BMPO, 200 μM of xanthine and 5 mM of deferoxamine in 50 mM phosphate buffer pH 7.4. The reaction was started by injection of xanthine oxidase to obtain 0.03 U/ml of the final enzymatic activity.

The effect of first and second fractions on the superoxide radical concentration was determined by the use of CPH as the trapping agent (Dikalov et al. 1997). The radical was generated with the same concentration of xanthine, XO and deferoxamine, but in the presence of 500 μM of CPH, instead of BMPO. The effect of addition of 26 mg/ml first fraction or second fraction on the CPH oxidation rate by superoxide radical was followed in time and compared the rate of the oxidation obtained in the presence of 200 U/ml of superoxide dismutase.

### EPR Measurements:

EPR spectra were recorded using Bruker EMX CW EPR spectrometer operating at X-Band (microwave frequency 9.85 GHz). Samples were measured in flat quartz EPR cells inserted into TM 110-type rectangular resonator from Radiopan (Poland). Parameters of the measurements were as follows: microwave frequency 9.85 GHz, modulation frequency 100 kHz, microwave power 5.97 or 21.97 mW (in the case of BMPO-O₂⁻), modulation amplitude 0.5 or 1 G (in the case of BMPO-O₂⁻), scan sweep width 100 or 80 G (in case of CPH measurements), time constant 20.48 ms, sweep time/spectrum 20.97 s, receiver gain 5.6·10³.

### Results:

### Hydroxyl Radical:

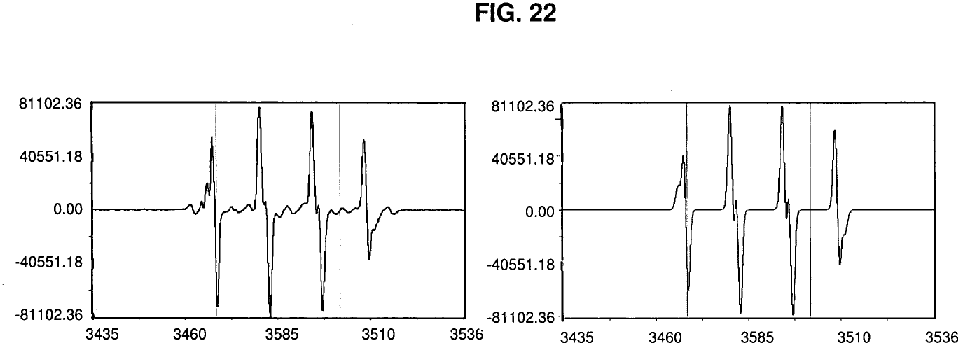

In the Fenton reaction (as well as in the Haber-Weiss reaction catalyzed by transition metals) the most reactive oxidant—hydroxyl radical is formed. This compound is prone to react with almost every encountered compound with the diffusion-limited rate constant (Bartosz 2003). Reaction of this radical with organic compounds usually leads to the formation of other derived radical. The EPR spectrum of hydroxyl-BMPO adduct is presented in FIG. 22.

As shown in FIG. 22, the spectrum can be satisfactorily simulated using following parameters: aN=14.16 G, aH=12.47 G, aH=0.62 G (53%), aN=14.16 G, aH=14.33 G, aH=1.21 G. These parameters are in very good agreement with those previously determined for BMPO-hydroxyl adduct (Zhao et al. 2001). One may conclude thereafter, that under this experimental conditions essentially only hydroxyl radical is formed.

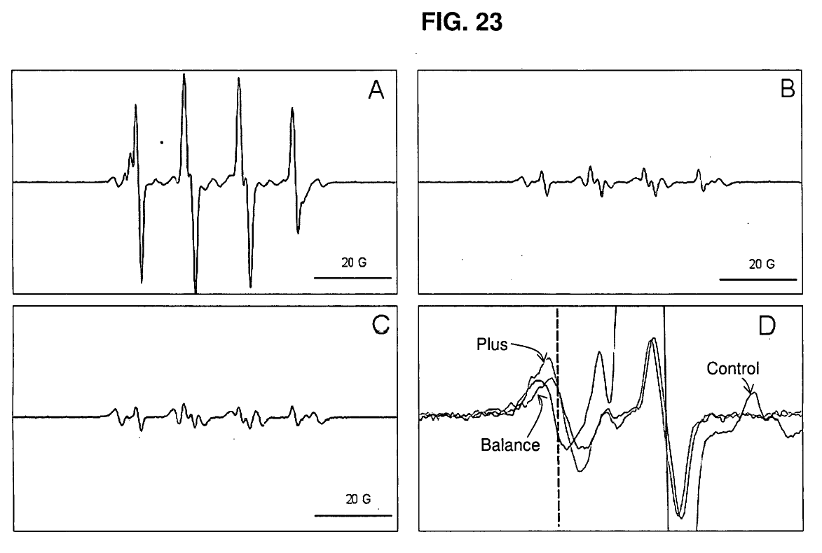

The intensity of the signal of hydroxyl-BMPO adduct in the absence and presence of the first or second fractions, each in the concentration of 26 mg/ml was compared. The results are presented in FIG. 23. When the reaction in conducted without any additions, the signal of the BMPO adduct is, by far, the most intense. However, addition of first fraction or second fraction to the reaction mixture causes a significant decrease in the signal intensity by 90 and 83% respectively (Table 9).

TABLE 9
Intensity of the BMPO*-OH EPR signal recorded in the absence
(Control) and presence of first fraction or second fraction.
Measurements were repeated 3 times.

| | | Percentage |
|---|---|---|
| Sample | Signal Amplitude [A.U.] | of the control |
| Control | 7.5 +/− 5.8 | 100% |
| First fraction (26 mg/ml) | 1.3 +/− 1.1 | 17% |
| Second fraction (26 mg/ml) | 0.73 +/− 0.13 | 10% |

The effect of the first and second fractions additions on the level of hydroxyl radical detected in this system seems to be predictable, if we take into account the fact that this radical reacts with all organic compounds. Therefore, first and second fractions act as a target for hydroxyl radical. However, in this system, no other derivative radicals are formed instead. In FIG. 23(D) one may clearly see that the level of line that comes from the BMPO-hydroxyl adduct are significantly decreased but signals of carbon centered radical are kept essentially at the same level for all samples. This suggests that first and second fractions are quite effective in scavenging of hydroxyl radical and prevent formation of derivative radicals.

### Carbon-Centered Radicals:

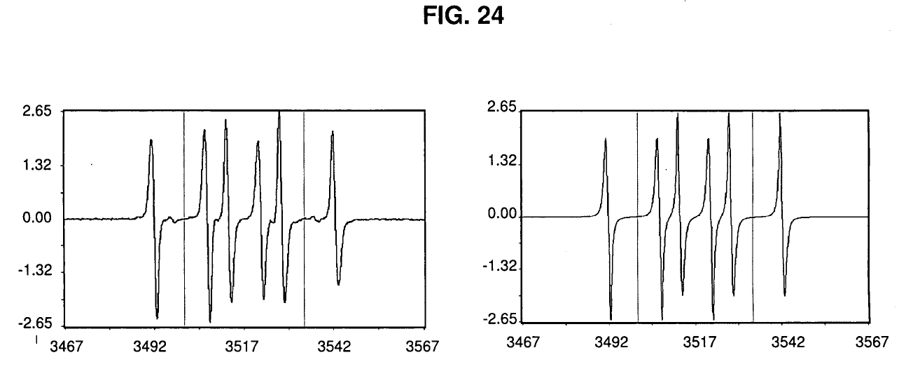

The effect of the first and second fractions on scavenging of carbon-centered radical was investigated. In this case, the appropriate radical was generated as a derivative compound, formed by rapid abstraction of β-hydrogen from ethanol molecule, induced by hydroxyl radical (see Materials and Methods). This kind of carbon radical, after reaction with BMPO, gives a stable nitroxide adduct that posses the characteristic spectrum presented in the FIG. 24A.

his spectrum was simulated (FIG. 24 B), yielding following parameters: aN=14.82 G, a_{H}=21.26 G (72%), a_{N}=14.82 G, a_{H}=19.97 G (27%), that are very close to the values typically observed for nitrone spin trap adduct with carbon centered radicals (Olive et al. 2000).

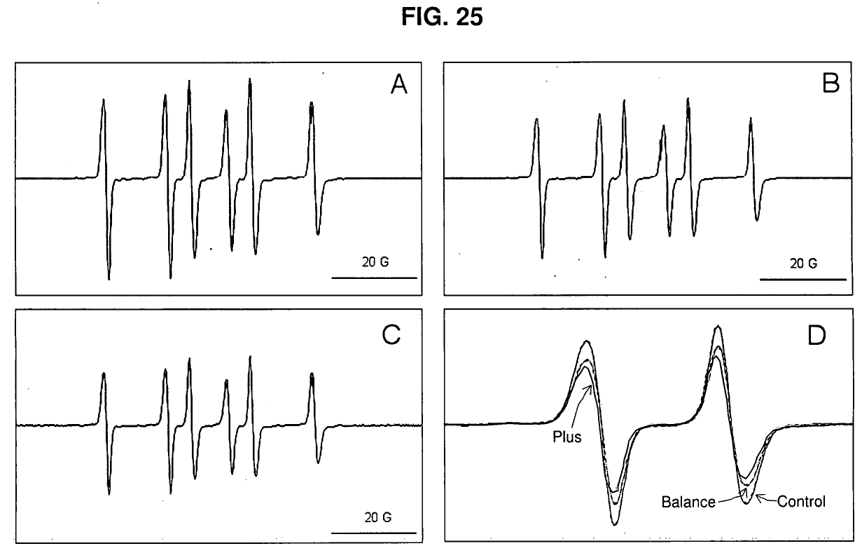

The amplitude of the signal intensity for carbon centered radical adduct with BMPO was measured in the presence of the first and second fractions and compared to the control sample, recorded in the absence of mentioned specimens (FIG. 25). Results are summarized in the Table 10. Upon addition of Second fraction to the reaction mixture generating the radical, signal is only slightly depressed, reaching approximately 74% of the control sample. The effect of the first fraction is essentially the same (71%), which induce the decrease of the quantity of trapped radical by the same extent as Second fraction. Such weak effect of the investigated peptides suggests that carbon centered radical is not effectively scavenged.

TABLE 10
Intensity of the BMPO adduct with carbon centered radical recorded in
the absence (control) and presence of the first fraction or second fractions.
Measurements were repeated 3 times.

| | | Percentage |
|---|---|---|
| Sample | Signal Amplitude [A.U.] | of the control |
| Control | 16.0 +/− 1.2 | 100% |
| First fraction (26 mg/ml) | 11.4 +/− 0.9 | 71% |
| Second fraction (26 mg/ml) | 11.9 +/− 1.8 | 74% |

### Azide Radical:

Although azide radical is not very important, from biological point of view, it has been reported to be formed in several enzymatic reactions (Kalayanaraman et al. 1984). It may be also generated by oxidation of N₃⁻ by hydroxyl radical.

This radical reacts quite selectively with tryptophan and in alkaline pH with cysteine and tyrosine amino acids, at the rate constant of the order of 10⁹ M⁻¹s⁻¹. Other aliphatic compounds and nucleic acids are relatively resistant to oxidation by azide radicals (Land and Prutz 1979).

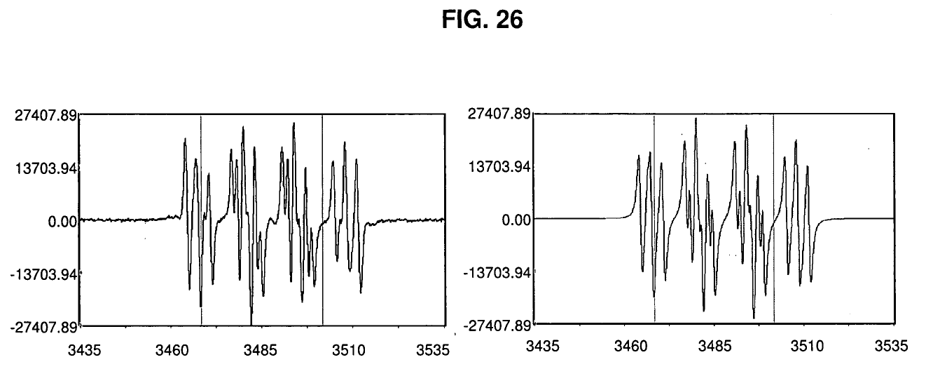

In this work, the azide radical was generated by oxidation of azide anion by hydroxyl radical. The EPR spectrum of BMPO-azide radical adduct is presented in FIG. 26. Although the spectrum of BMPO*-N₃ has not been previously reported, it was successfully simulated assuming that 88% of trapped radical comes from azide adduct (aN=13.96 G, aN^{azide}′=3.15 G, aH=12.7 G) and remaining 12% from hydroxyl adduct (see parameters for hydroxyl-BMPO adduct).

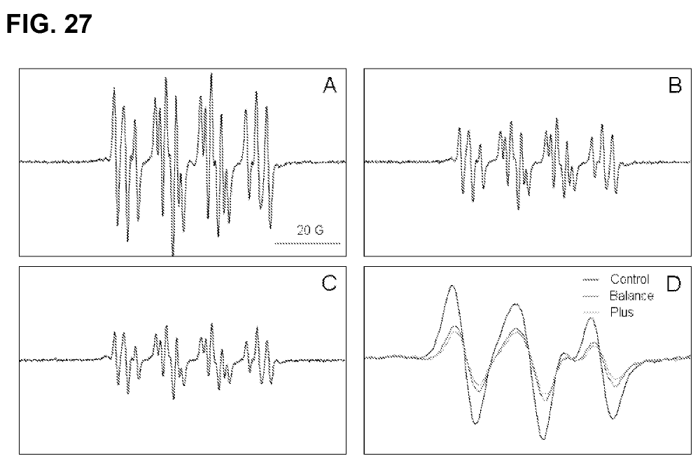

This indicates that azide radical is not the single compound present and some quantities of hydroxyl radical is also trapped by BMPO. The EPR spectra of BMPO-azide radical recorded in the absence and presence of the first and second fractions are presented in FIG. 27. It is evident that addition of the first fraction or second fractions decreases the amplitude of the detectable azide radical by 58 and 64% respectively (Table 11). Shape of the EPR spectrum recorded in the absence and presence of the first fraction or second fraction is not changed, suggesting that proportion between hydroxyl and azide radical are kept in the same proportions as in the control sample.

TABLE 11
Intensity of the BMPO adduct with azide radical recorded in the
absence (control) and presence of the first fraction or second fraction.
Measurements were repeated 3 times.

| | | Percentage |
|---|---|---|
| Sample | Signal Amplitude [A.U.] | of the control |
| Control | 12.1 +/− 0.9 | 100% |
| First fraction (26 mg/ml) | 4.4 +/− 0.2 | 36% |
| Second fraction (26 mg/ml) | 5.1 +/− 0.5 | 42% |

The results of spin trapping of azide radicals show that the investigated peptides are able to scavenge significant amount of azide radical. However, these scavenging properties are less profound than in the case of hydroxyl radical but somewhat higher than towards carbon centered radical.

### Cumene Hydroperoxide-Derived Radicals:

The EPR spectra of adducts of BMPO with alkoxyl radicals are not known. What is more, spectra generated by spin trapping of radicals that come from decomposition of organic hydroperoxide are usually very complicated (Chamulitrat et al. 1989, Dikalov and Mason 2001). Addition of Fe²⁺ ions to the solution of cumene hydroperoxide should generate large amount of alkoxyl radicals, as well as the other derivative radicals including peroxyl and carbon centered radicals (Bartosz 2003).

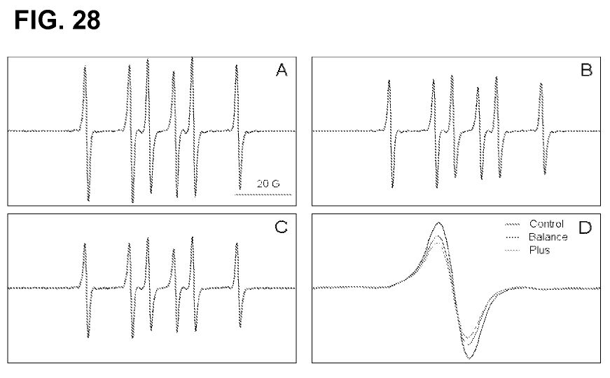

In case of the system used in this work, the only radical that is trapped by BMPO after addition of iron to cumene hydroperoxide solution, is clearly carbon centered radical (FIG. 28), whose spectrum is essentially identical to that presented in FIG. 24.

Effect of the first and second fractions on the amplitude of the radical generated in this case is summarized in Table 12. The results are very similar to those obtained for carbon-centered radical described previously, indicating that both of the investigated specimens are not very effective in scavenging of this radical.

TABLE 12
Intensity of the BMPO adduct with cumene hydroperoxide-derived
radical generated by one-electron reduction of OOH moiety
by Fe²⁺ in the absence (Control) and presence of the first
fraction or second fractions (measurements were repeated 3 times).

| | | Percentage |
|---|---|---|
| Sample | Signal Amplitude [A.U.] | of the control |
| Control | 6.5 +/− 3.1 | 100% |
| First fraction (26 mg/ml) | 4.2 +/− 2.5 | 64% |
| Second fraction (26 mg/ml) | 4.8 +/− 1.3 | 73% |

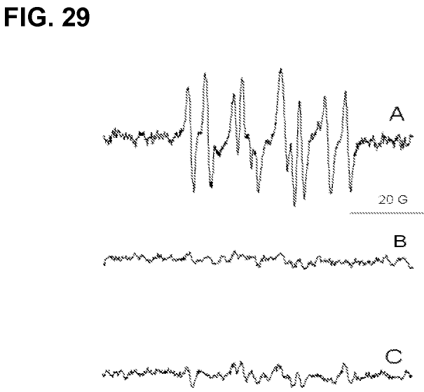

Beside the ferrous ion-induced generation of the radicals from cumene hydroperoxide, the effect of spontaneous decomposition of this compound was studied. If the high concentration of cumene hydroperoxide (8% in this case) is added to phosphate buffer solution of BMPO spin trap, the spontaneously generated radical is trapped, giving the composite spectrum presented in FIG. 29 A.

However, if the process is carried out in the presence of the first fraction or second fractions (26 mg/ml) the EPR signal is completely abolished (FIGS. 29 B and C, respectively) and there is no detectable EPR spectrum. This result indicates that radicals derived from metal-catalyzed cumene hydroperoxide slow decomposition are effectively scavenged by the investigated peptide preparations, decreasing the BMPO adducts by 100%.

### Superoxide Radical:

The last radical that was taken into account in this work is superoxide anion. Even though superoxide radical is relatively not reactive, it undergoes quite fast dismutation, what in the presence of trace amounts of transition metals, leads to the formation of hydroxyl radical and therefore triggers the cascade of oxidation of biomolecules and membranes.

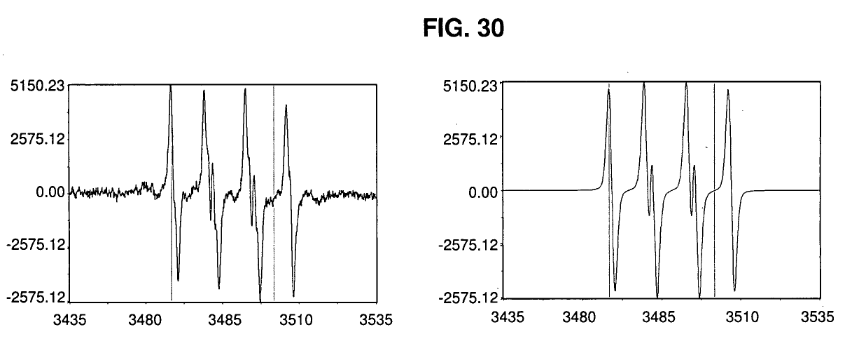

During in vitro studies of superoxide radical, especially in water, the effect of metals must be carefully controlled. Therefore, the additions of strong chelators like deferoxamine are used to prevent formation of hydroxyl radical from superoxide anion. In this work, for the superoxide radical generated by xanthine/xanthine oxidase in the 50 mM phosphate buffer, addition of 5 mM of deferoxamine completely prevents formation of derivative *OH radical. Indeed, the EPR spectrum of BMPO adduct created in this system shows, that only superoxide radical is trapped (FIG. 30 A). The experimental EPR spectrum of BMPO*-OOH was simulated giving the following parameters: aN=13.368 G, aH=11.049 G. These parameters are essentially identical to previously reported (Zhao et al. 2001; Keszler et al. 2003).

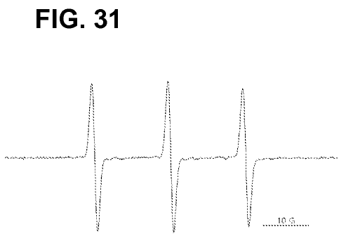

The use of classic nitrone spin traps (BMPO, EMPO, DMPO, etc.) is disadvantageous due to the very slow reaction with superoxide radical, short lifetime of the adduct as well as tendency to hydrolysis (DMPO). This often compromise the measurement of superoxide radical production. Therefore in this work hydroxylamine CPH was used to monitor the amount of superoxide radical (Dikalov et al. 1997; Fink et al. 2000). The product of the oxidation gives rise to the formation of extremely stable nitroxide spin label, detectable by EPR spectroscopy (FIG. 31).

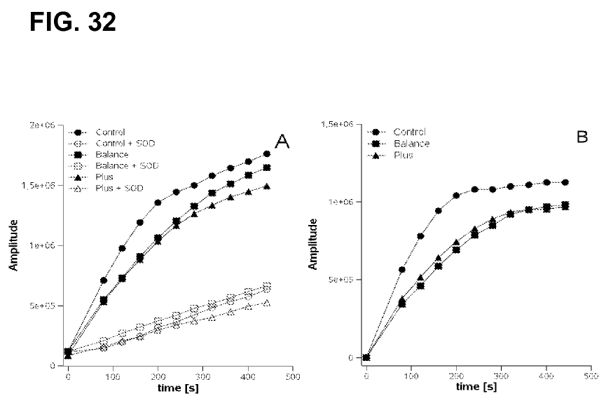

However, the major drawback of this compound is, that its EPR spectrum does not depend on the radical that induced oxidation of the hydroxylamine. Therefore, in the case of study of superoxide radical formation it is necessary to measure oxidation rate in the presence of superoxide dismutase to exclude other than superoxide sources of CPH oxidation. In FIG. 32 A, the rates of oxidation of CPH by superoxide radical measured in the absence or presence of the first fraction or the second fractions are presented.

The effect of the peptide preparations was estimated by the comparison of the rates measured for the control and the respective peptide preparation, using linear regression for initial 4 points. The results are shown in Table 13. It is clearly seen, that the rate of oxidation of CPH by superoxide radical is the highest, if the reaction is carried out without addition of the peptides. The rate of oxidation is somewhat decreased if the first fraction or the second fraction is added to the reaction mixture.

To test the effect of oxidation of the hydroxylamine, which is not caused by superoxide radical, the same reaction was followed in the presence of SOD (200 U/ml). No significant difference in the non-radical mediated oxidation rate of CPH in the absence and presence of the first fraction or the second fraction was observed (FIG. 32(A), open symbols). The rates obtained after subtraction of background oxidation of the hydroxylamine are presented in FIG. 32 (B).

TABLE 13
The rates of CPH oxidation by superoxide anion in the absence (control)
and presence of the first fraction or the second fraction: rates were
estimated as slope obtained from the linear fit to the first 4 points
of the curves shown in FIG. 32 (B).

| | | Percentage |
|---|---|---|
| Sample | CPH oxidation rate [A.U.] | of the control |
| Control | 5.9 +/− 0.5 | 100% |
| First fraction (26 mg/ml) | 4.0 +/− 0.3 | 67% |
| Second fraction (26 mg/ml) | 3.9 +/− 0.3 | 66% |

### In Summary:

In this work, the influence of two therapeutic peptide preparations: the first fraction and the second fraction on in vitro production and scavenging of free radicals, using EPR spin-trapping technique was studied. Antioxidant properties towards hydroxyl, azide, carbon centered, cumene hydroperoxide derived and superoxide radicals were measured by competition with the reactivity of the radicals with BMPO or CPH. In the view of the results, it is concluded that both of the investigated samples reacts differentially with produced radicals.

However, the effect of Second fraction seems to be essentially identical with the effect obtained in the presence of first fraction. This observation suggests that the first fraction or the second fraction reacts with the investigated radicals in the very similar manner, decreasing their concentrations almost by the same extend. It should be noted that some radicals (especially hydroxyl and azide) are quenched by more than 50%, whereas carbon centered radical is only weakly affected by both the first fraction and the second fraction. Surprisingly strong effect is observed when the investigated samples are added to the mixture containing cumene hydroperoxide. Its slowly decomposition gives rise to the formation of several different radical species that are easily detected by BMPO spin trapping. However, when the first fraction or the second fraction is added to the mixture, the EPR signal of the radicals is almost completely abolished. This suggests that if organic hydroperoxides undergoes slow spontaneous decomposition, the formed radicals are effectively eliminated by both, the first fraction and the second fraction.

In the case of superoxide radical the first fraction and the second fraction also exhibit some scavenging properties, decreasing the level of the radical detected by CPH. However, such decrease (by approx. 33-34%) is not especially profound being very similar to the results obtained by measurement of carbon centered radical.

In summary, one may find that in the presence of the first fraction and the second fraction all kinds of radicals are somewhat consumed. Products of decomposition of cumene hydroperoxide are essentially completely scavenged, whereas the other types of radicals are eliminated less profoundly. The effectiveness of the first fraction and the second fraction decreases in the order: hydroxyl radical>azide radical>superoxide radical>carbon-centered radical. It is also concluded that carbon centered radicals are rather resistant to scavenging by the first fraction and the second fraction.

### Part 3: Biological Functions of the First Fraction and the Second Fraction Through In Vivo Experiments on Animals

I. Anti-Allergic Functions of the First Fraction in Rats with OVA-Induced Pleurisy

A constantly increasing incidence of allergic diseases in the last few decades represents an aggravating problem of the contemporary civilization, deteriorating the quality of life. In an attempt to prevent and treat that malfunction of the immune system new therapeutic strategies have been developed including subcutaneous specific immunotherapy and sublingual immunotherapy (SLIT).

In a study conducted, the peptide-treated (with first and second fractions) mice showed: some decrease of the leukocyte number, the analysis of blood picture revealed significant increases of eosinophil (2.8-fold) and macrophage levels (7.2-fold) and a significant rise in the band forms leading to a conclusion that the first and second fractions may be used as anti-allergents.

Further, in a study including Real Time PCR technique analysis of Foxp3 gene expression by Peyer's patches derived from mice with OVA-induced allergy it was found that; total RNA was isolated from Peyer's patches with good yield of capacity and quality; all selected pair primers for PCR and qPCR techniques showed satisfying reactivity and specificity; there were no unspecific or doubled products in the PC reaction; applied qPCR technique occurs adequate and accurate for successful Foxp3 gene expression determination; no visible differences exist in Foxp3 and TGF-β gene expressions by individual sample; and visible decrease of both Foxp3 and TGF-β gene expressions were noted for samples derived from “AB” animal group. Such findings conclude that the peptide compositions have anti-allergic properties.

Also, in a study conduced in rats with OVA-induced pleurisy-Histopathological examination, it was found that the peptide compositions have anti-inflammatory effects.

### II. Suppression of Myelogenesis by Second Fraction in Healthy Female Rats (The Decrease in WBC and Neutrocytes in Female or Male+Female Rats Respectively)

### Safety/Toxicity Study of the Second Fraction on a Rat Model

Peptide preparations are known as immunomodulators of the cellular and humoral response in the organism. In a study conducted, it was found that no doses of the second fraction elicited any pathologic lesions in lungs, liver, kidney and stomach of rats. However, in the jejunum, at higher doses (e.g., 0.20571 mg), the second fraction caused moderate shortening and deformations of intestinal villi and increased a crypt depth. It suggests a dose-dependant cytotoxic effect on intestinal enterocytes. In hematological examinations, the second fraction revealed a decrease in the white blood cells number mainly due to the depletion in the neutrocytes' number. The toxic influence of the second fraction was found to be very weak even at high doses.

For example, the second fraction administered in the amount of 10× maximum single dose for rats (MSD_{R}=0.01029 mg) three times daily for five days elicited, in comparison to the parameter values for control rats, a 2.84% decrease in the height of the small intestine villi (HV), a 4.51% increase in the depth of the small intestine crypts (DC), a 9.03% decrease in the white blood cells number (WBC), and a 5.57% decrease in the number of neutrocytes (N) but only a 0.65-0.95% decrease in the number of red blood cells (RBC), a 0.41-0.64% in the hemoglobin concentration (Hb), a 0.80% in hematocrit (Ht), and a 2.10% decrease in the platelet number (Pl), whereas, at a dose of 1×MSD_{R}, changes were equal to 0.29% HV, 0.44% DC, 0.94% WBC and 0.57% N, 0.06-0.09% RBC, 0.04-0.06% Hb, 0.08% Ht and 0.21% Pl.

### III. Stimulation of Myelogenesis (the Increase in WBC and Neutrocyte Proliferation in Healthy Female or Male+Female Rats, Respectively) and the Decrease in Basophil and Eosinophil Number by First Fraction in Healthy Male+Female Rats

### Safety/Toxicity Study of the First Fraction on a Rat Model

In a study conducted to assess potential pathologic effects of the first fraction on rats, it was found that the first fraction did not elicit any pathologic lesions in lungs, liver or kidney. However, a moderate increase in the mucous secretion was observed in stomach at higher doses (e.g., 0.57143 mg) of the first fraction which suggests an irritating influence of these doses on the gastric mucosa. In the jejunum, the first fraction caused a noticeable linear increase in the intestinal villi height and the crypt depth. Hematological examinations revealed that the first fraction caused an increase in the number of white blood cells mainly due to the increase in the neutrocytes' number. The first fraction elicited changes in other examined parameters as well, however, these effects were found to be very week even at high doses.

For instance, the first fraction that was administered in the amount of 10×MSD_{R} three times daily for five days, elicited, in comparison to the control rats, a 1.53 (in male rats) or 1.19% (in female rats) increase in the height of the small intestine villi (HV), a 3.62% increase in the depth of the small intestine crypts (DC) in male rats, a 9.00% increase in the white blood cells number (WBC) in female rats, and a 5.02% increase in the number of neutrocytes (N) in the both genders as well as 2.29% decrease in the number of red blood cells (RBC), and 1.93% in the hemoglobin concentration (Hb), a 2.42% in hematocrit (Ht), and a 3.29% increase in the platelet number (Pl)—all in female rats, and a rather high drop (by 51.84%) in the number of basocytes, whereas, at a dose of 1×MSD_{R}, changes in both genders or solely in female rats were equal to 0.12-0.15% in HV, 0.33-0.36% in DC, 0.87% in WBC and 0.50% in N, 0.23% in RBC, 0.19% in Hb, 0.24% in Ht and 0.33% in Pl; an exception is again a 7.05% decrease in the number of basocytes.

### Useful Forms of the Bioactive Peptide Compositions According to the Present Invention

Generally, the bioactive compositions according to the present invention may be administered to a patient in various forms, including an injectable liquid form injected into a patient, a sublingual form (drops) placed beneath a patient's tongue, as a food supplement form which would be ingested by the patient, a topical ointment/cream which would be applied externally to a patient's skin, etc.

As a food supplement form, the liquid bioactive peptide composition formulated in a manner as discussed above, may be freeze dried and then bound together with a combination of concentrated elderberry and blueberry extract, selenium and magnesium. Also, as a food supplements, the bioactive peptide compositions may be may be freeze dried and bound together with a combination of various desirable binding components, such as blueberry, xylitol, vitamin C and E. Further, nasal sprays and topical sprays may be formulated using the peptide compositions of the present invention.

A topical ointment or cream having the bioactive peptide composition is effective for treating acne, burned skin, a cosmetic additive, etc. Such cream may be effectively used to treat diaper and various skin rashes and irritations, chemical sensitivities, insect bites, inflammed or itching skin, rapid would healing, etc.

### Highlights of Mechanisms of the Anti-Allergic/Anti-Inflammatory Action of the Second Fraction Peptide Composition

The main experimental findings, being statistically significant (p<0.05), which were measured in the rats with acute allergic disease after the oral administrations of the second fraction are as follows:
Second fraction reduced the level of eosinophils in blood, lungs, and peritoneal cavity of allergic rats, restoring its level either entirely (100%) or in great part (62-83%).
Second fraction reduced the level of macrophages in the pleural cavity and lungs of the allergic rats, restoring its level entirely.
Second fraction reduced concentrations of the main proinflammatory Th2-type cytokines IL-4 in splenocytes and IL-5 in the pleural cavity fluid, restoring their values either entirely or almost entirely (89%), respectively.
Second fraction reduced totally the overall inflammatory symptoms, being represented by the humoral response (HR).
Second fraction elevated the level of monocytes in lungs and the peritoneal cavity fluid, restoring its values entirely.
Second fraction elevated the concentration of the important anti-inflammatory cytokine IL-10 in splenocytes and blood by 21-24%.
Second fraction elevated concentrations of the main Th1-type cytokine IFN-γ in splenocytes (6020%), serum (122%) and BAL (74%).

At the increased number of lymphocytes in the pleural cavity (by 123%), blood (15%), lungs (10%) and the peritoneal cavity (19%) of the Second fraction-treated allergic rats, the above-mentioned experimental findings lead to the conclusion that the number of Th2 cells was reduced whereas the number of Th1 cells increased.

This conclusion is possible in the situation, in which it was experimentally proven that the oral administration of the second fraction did not induce the production of regulatory CD4⁺CD25⁺ T cells. From the lymphocyte subpopulations involved here, also Th17 cells can be excluded because the hypothetical second fraction-stimulated production of Th17 cells from naïve Th cells requires the enhancement of the TGF-β level in allergic rats, which did not occur; besides due to the small percentage (1-10%) of Tγδ cells the contribution of product, Th17, to the increased number of lymphocytes would be also small.

Moreover, the highly increased level of IFN-γ, which was caused by the administration of second fraction peptide composition downregulates T cell differentiation into Th17 cells. In consequence, one can conclude that the cellular response (CR) increased, mainly due to the increase of the number and activity of Th1 cells. Moreover, owing to the cross-linking between Th1 and Th2 cells, the HR decreased.

The experimental findings together with the latest conclusions determine the mechanisms through which second fraction peptide composition affects the impaired immune system. It was found that:
Th2 cells produce IL-13 through which Th1 cells are inhibited. This function of Th2 cells was inhibited by Second fraction in the extensive manner (because the percentage of eosinophils was reduced by second fraction peptide composition).
Th2 cells contribute to the HR through IL-13 and through the stimulation of B cells followed by the stimulation of mast cells and basophils, resulting in the secretion of proinflammatory mediators. These functions of Th2 cells were inhibited by second fraction peptide composition in the extensive manner.
IL-4 and IL-5 stimulate B cells to produce IgE, which is followed by the stimulation of mast cells and basophils, which leads to the HR. Due to the lowered production of IL-4 and IL-5, the stimulation of B cells was inhibited by second fraction peptide composition. Consequently, the HR was inhibited.
Eosinophils secrete IL-5 that is their autocrine agent (a positive feedback). The second fraction peptide composition inhibited this secretion in the extensive manner by reducing the percentage of eosinophils, which was accompanied by the reduced level of IL-5.
Eosinophils are attracted to the site of inflammation and stimulated by Th2 cells through VCAM-1 and GM-CSF, and IL-3, respectively. Second fraction stopped the highly developed process of eosinophilia, which can result not only from the measured inhibition of eosinophil proliferation and activity but also from the lowered attraction and migration of these cells to lungs and peritoneal/pleural cavity, which can—in turn—result from the lowered production of integrins, VCAM-1, chemokines (e.g. CCL3/5/7/13).
IL-4 is an autocrine agent for Th2 cells (a positive feedback). IL-4 is also secreted by eosinophils, which enhances the above autocrine effect of IL-4 towards Th2 cells. Second fraction inhibited the self-stimulation of Th2 cells by IL-4 in the extensive manner, resulting from the lowered percentage of eosinophils and Th2 cells.
Eosinophils contribute to the HR through toxic proteins (ECP, MBP, EPO), leukotriene LCT4 and prostaglandins PGE1 and PGE2. Second fraction stopped entirely or almost entirely the HR; in consequence, the levels of these toxins were diminished to inoffensive values.
Eosinophils inhibit Th1 cells through the enhancement of the kynurenine production. Eosinophils, which secrete IL-12, are also capable of stimulating the production of Th1 cells from naïve Th cells. These two mutually antagonistic functions of eosinophils were annihilated by Second fraction as the activity of eosinophils was blocked by Second fraction.
IFN-γ is an autocrine agent for Th1 cells; it stimulates MHC II expression on these cells. Moreover, IFN-γ inhibits both the proliferation and activity of Th2 cells via an intracellular protein SOCS-1 (suppressor of cytokine signaling-1). Due to the huge secretion of IFN-γ caused by the oral administration of Second fraction, all these effects are highly enhanced.
IFN-γ and Th1 cells contribute, directly or via cytokines, respectively, to the cellular response (CR). Second fraction stimulated and enhanced this process.
Indirect contribution to the CR reveals also macrophages that produce IFN-γ. However, this contribution is relatively small in comparison to Th1, NK and NKT cells. Since Second fraction reduced the level of macrophages, it is reasonable to expect that the contribution of macrophages to the IFN-γ production was lowered by second fraction. However, the IFN-γ production increased, which means that macrophages did not play a role of the main producer of IFN-γ. It is possible that second fraction also stimulates activities of NK and NKT cells, which needs, however, experimental confirmation.
Monocytes, macrophages and Th2 cells produce IL-10, which inhibits Th1 cells and eosinophils. Owing to the Second fraction-reduced levels of Th2 cells and macrophages, the overall production of IL-10, even at the elevated level of monocytes, is only slightly elevated and does not lead to the inhibition of Th1 cells due to the strong and opposite stimulatory effects initiated by Second fraction.

The above interactions are accomplished via pattern recognition receptors (such as TLR) located on surfaces of macrophages, monocytes, Th1 and Th2 cells shown in FIG. 21, and via cytokines, eotoxins, adhesive molecules and chemoattractants. Moreover, an interplay of the non-specific and specific immune systems is important at the beginning of allergy.

At the beginning phase of allergy, different TLRs (TLR4 in particular) positioned on epithelial and endothelial cells, dendritic and mast cells (in oral, nasal, airway and intestinal mucosa, in general) are implicated. These cells, when activated by an antigen, secrete various chemokines (e.g. CCL2/3/5/8), cytokines (IL-1/6/12/18, TNF) and other agents (histamine, PAF, PGD, LTB, adhesive molecules), which attract a spectrum of effector cells (e.g. dendritic cells, macrophages and T cells at last). They, in turn, secrete cytokines to attract and activate other cells (like neutrophils being activated by IL-8 and GM-CSF).

Finally, many MHCII/I-equipped cells (monocytes, B cells, dendritic cells, epithelial and endothelial cells, keratinocytes) present the antigen to the T cell via a TCR to start the specific immune response.

Potentially, there exist several targets-cellular receptors in the immune system for second fraction peptide compositions. The first possible target is the beginning step of the allergic non-specific response, in which the second fraction peptide compositions behave like the so-called altered peptide ligands that generate an inhibitory or none signal instead of a stimulatory one within epithelial cells in oral, nasal, esophagus, airway mucosa.

At this stage, the formation of the allergen-TLR4 complex can be blocked by the competitive formation of the Second fraction peptide-TLR4 complex, and, in consequence, the non-stimulated cells do not secrete cytokines and proinflammatory mediators, and other signal molecules that play a role of attractors for neutrophils, monocytes, macrophages and T cells, finally.

Also, it is likely that second fraction peptide compositions after binding with the TLR4 on antigen-presenting cells selectively promote the secretion of IL-12, which, in turn, selectively stimulates the development of Th1 cells from naïve Th cells.

T cell receptors, TCR, which bind peptide antigens (produced by enzymatic hydrolysis of proteins), being linked with MHC molecules located on antigen presenting cells, are capable of binding second fraction peptide compositions. Since the change even a single amino acid in the antigen can result in inhibition instead of stimulation of the recognizing T lymphocyte, it is very likely that the specific proinflammatory immune reaction can also be blocked by second fraction peptide composition. This effect explains the observed inhibition of Th2 cells.

Simultaneously there exists, the strong stimulation of Th1 cells, which indicates that certain second fraction peptide composition behave like the so-called superagonist for TCR on these subclass of helper lymphocytes. During the second fraction peptide composition presentation to the TCR, short peptides (composed of less than 10 AA residues) can be presented by those cells that have the MHC I molecule, whereas longer peptides (having circa 10-20 AA residues) require cells with the MHC II molecule.

Accordingly, based on foregoing, it may be concluded that second fraction peptide composition is a powerful antiallergic and anti-inflammatory agent which acts in the manner characteristic for conventional specific immunotherapy (1-5) that was designed to decrease IL-4 and IL-5 cytokine production by CD4⁺ T cells accompanied by a shift towards increased IFN-γ production, thus to shift the Th2-type cytokine production to the Th2-type one by lymphocytes. It can be easily seen that second fraction peptide composition produced such effects in the allergic rats.

Such conclusion may be perceived as unexpected results in which, T cell epitopes amongst the second fraction peptide compositions. Therefore, either the variety of peptides in second fraction is so huge that also those epitopes exist in second fraction or the effect of bystander immune response really occurs.

### Illustrative Guidelines on Serving Products Having First Fraction and Second Fraction Peptide Compositions

A tablet which includes the first and/or second fraction peptide compositions as active ingredient in the amount of 2.5 mg/tab may be administered once a day for modulating immunity. It may be necessary to consult patient's health care professional to monitor progress of immunity development during treatment period of administering such tablet. Children may be administered with a half of the tablet daily.

### Illustrative Guidelines on Serving Amounts of First Fraction Peptide Composition

The following dosages of the first fraction peptide composition for treating the following ailments are provided as a guideline only. The amount of dosages, illustrated below for adults, may be changed based on further studies. Children may be administered with half of the adult serving dosage specified below.

The amount of dosage may be changed based on further studies and/or feedback from the patients. Each tablet of the first fraction peptide composition includes about 4 mg of the peptide composition.

It may be necessary to consult patient's health care professional to monitor progress during treatment using the first fraction peptide composition.
Flu: 4 tablets be administered twice daily for 4 days or until symptoms are gone.
Mononucleosis: 3 tablets may be administered twice daily for 12 days or until symptoms are gone.
Epstein-Barr: 3 tablets may be administered twice daily for 30 days or until symptoms are gone.
HIV: 4 tablets may be administered twice daily.
Hepatitis: 4 tablets administered twice daily.
Cancer: 4 tablets may be administered twice daily.
HPV & Warts (Human papillomavirus—the cause of warts, plantar warts, genital warts and a precursor of cervical cancer): 3 tablets may be administered twice daily at first sign of onset, and may be continued until symptoms are gone.
Malaria: 4 tablets may be administered twice daily until symptoms are gone.
Herpes: 3 tablets may be administered twice daily at first sign of onset, and may be continued until symptoms are gone.
Dengue Fever: 4 tablets may be administered twice daily or until symptoms are gone.

### Illustrative Guidelines on Serving Amounts of Second Fraction Peptide Composition

The following dosages of second fraction peptide composition for treating the following ailments are provided as a guideline only. The amount of dosages, illustrated below for adults, may be changed based on further studies. Children may be administered with half of the adult serving dosage specified below.

Each tablet of the second fraction peptide composition includes about 1.44 mg of the peptide composition. It may be necessary to consult patient's health care professional to monitor progress during treatment using the second fraction peptide composition. It is advisable not to administer more than 8 tablets of the second fraction peptide composition (1.44 mg/tablet) per day to a patient.
Allergies: 2 tablets may be administered twice a day until symptoms are gone, and then may be used as needed.
Asthma: 2 tablets may be administered twice a day until symptoms are gone.
Arthritis: 1 tablet may be administered twice a day until symptoms are gone.
Severe Arthritis: 2 tablets may be administered twice a day until symptoms are gone.
Multiple Sclerosis: the second fraction peptide composition may be administered to patients having multiple sclerosis.

### Illustrative Guidelines on Applying of Renewity Skin Care Product Having Second Fraction Peptide Composition

An effective therapeutic anti-inflammatory, burn and wound healing cream may be formulated having the first fraction and/or second fraction peptide compositions as active ingredients. Such anti-inflammatory, burn and wound healing cream (also referred as a renewity skin care product) may be applied to help accelerate skin regeneration. In order to particularly treat burns, wounds, lacerations, acne, various types of skin rashes, the anti-inflammatory, burn and wound healing cream may be gently applied forming a thin layer on affected area(s) after showering, or washing affected area(s). It is recommended to apply the anti-inflammatory, burn and wound healing cream to the affected areas minimum twice daily. It is also recommended to continue to use until the symptoms are disappeared, then use as needed.

Although the present invention has been described herein with respect to a number of specific illustrative embodiments (examples), the foregoing description is intended to illustrate, rather than to limit the invention. Those skilled in the art will realize that many modifications of the illustrative embodiment could be made which would be operable. All such modifications, which are within the scope of the claims, are intended to be within the scope and spirit of the present invention.

What is claimed is:

1. A bioactive peptide composition consisting essentially of a mixture of:
serum;
albumin;
casein;
peptone;
a digestive enzyme; and
a base.

2. A bioactive peptide composition according to claim 1, wherein the digestive enzyme includes at least one of a pancreatin supplying amylase, protease, lipase, protease II, or protease III, aylase, lactase, and lipase.

3. A bioactive peptide composition according to claim 1, wherein for every 50 ml of serum said mixture includes 1-2 g albumin; 10-50 g casein; 10-30 g peptone; 0.1-0.1 g digestive enzyme; and 5-10 g base.

4. A bioactive peptide composition according to claim 1, wherein said peptide composition includes molecules having a molecular weight greater than 10 kDa; and wherein molecular mass of said peptide composition, evaluated by a using chromatographic bands recorded by SDS-PAGE electropherosis, ranges between 3000 Da and 35000 Da.

5. A bioactive peptide composition according to claim 4, said peptide composition has a protein content of about 10.5 μg/μg obtained at absorbance at 280 nm; and is about 2.5 μg/μg according to bicinchoninic acid assay method.

6. A bioactive peptide composition according to claim 4, wherein said peptide composition includes a number of amino acid residues in a range of 27 and 315.

7. A bioactive peptide composition according to claim 4, wherein said peptide composition exhibits one or more of a free radical scavenging property, an antioxidative property, an enzymatic activity of SOD in human blood and of an enzymatic activity GPx in human erythrocyte.

8. A bioactive peptide composition according to claim 1, wherein said peptide composition includes molecules having a molecular weight less than or equal to 10 kDa; and wherein molecular mass of said peptide composition, evaluated by a using chromatographic bands recorded by SDS-PAGE electropherosis, ranges between 3000 Da and 6000 Da.

9. A bioactive peptide composition according to claim 8, wherein said peptide composition has a protein content of about 8 μg/μg obtained at absorbance at 280 nm; and is about 1.5 μg/μg according to bicinchoninic acid assay method.

10. A bioactive peptide composition according to claim 8, wherein said peptide composition includes a number of amino acid residues in a range of 27 and 54.

11. A bioactive peptide composition according to claim 8, wherein said peptide composition exhibits one or more of a free radical scavenging property, an antioxidative property, an enzymatic activity of SOD in human blood and of an enzymatic activity GPx in human erythrocyte.

12. A bioactive peptide composition according to claim 8, wherein said peptide composition at 16.5% volume/volume concentration enhances activity of superoxide dismute and glutathione peroxide in human blood cells.

13. A bioactive peptide composition according to claim 1, wherein said peptide composition includes a sequence having 59 amino acid residues, selected from the group consisting essentially of:
GlyGluProGlyAlaAspLysSerThrProMetPheValLeuGlnHis
ArgProIleGluThrGlySerLysTyrGluAlaAspArgPheGlnPro
ValAlaIleGlyAsnLysArgGlnAlaLeuGlnGlyProValGluArg
LeuGluLysProGlyPheTyrLeuProGlnLys;
GlyGluProGlyAlaAspLysSerThrProMetPheValLeuGlnHis
ArgProIleGluThrGlySerLysTyrGluAlaAspArgPheGlnPro
ValAlaIleGlyAsnLysArgGlnAlaLeuGlnGlyProValGluArg
LeuGluLysProGlyPheIyrLeuProGlnGlu;
GlyGluProGlyAlaAspLysSerThrProMetPheValLeuGlnHis
ArgProIleGluThrGlySerLysTyrGluAlaAspArgPheGlnPro
ValAlaIleGlyAsnLysArgGlnAlaLeuGlnGlyProValGluArg
LeuGluLysProGlyPheTyrLeuProGlnAsn;
AlaGluProGlyAlaAspLysSerThrProMetPheValLeuGlnHis
ArgProIleGluThrGlySerLysTyrGluAlaAspArgPheGlnPro
ValAlaIleGlyAsnLysArgGlnAlaLeuGlnGlyProValGluArg
LeuGluLysProGlyPheTyrLeuProGlnAsn;
and other similar peptide sequences in which the terminal residues are varied, and in which the 6^{th}-59^{th} residues are ordered randomly.

14. A bioactive peptide composition according to claim 1, wherein said peptide composition includes a sequence having 27 or 41 amino acid residues, selected from the group consisting essentially of:
GlyGluProAlaProValLeuGlyGlnAlaValPheProSerThrLeu
GluLysArgTyrIleAspArgGlyGlnAsnLys;
GlyGluProAlaProValLeuGlyGlnAlaValPheProSerThrLeu
GluLysArgTyrIleAspArgGlyGlnAsnGlu;
GlyGluProAlaProValLeuGlyGlnAlaValPheProSerThrLeu
GluLysArgTyrIleAspArgGlyGlnLysAsn;
AlaGluProGlyProValLeuGlyGlnAlaValPheProSerThrLeu
GluLysArgTyrIleAspArgGlyGlnAsnGlu;
AlaProGlyAlaProPheAsnLysValThrGluMetLeuAspArgTyr
SerIleGlyGlnHisGluArgValProLeuGlyPheGlnGluIleLys
SerGlyProGluLysGlnArgGlnLeu;
AlaProGlyAlaProPheAsnLysValThrGluMetLeuAspArgTyr
SerIleGlyGlnHisGluArgValProLeuGlyPheGlnGluIleLys
SerGlyProGluLysGlnArgGlnLys;
AlaProGlyAlaProPheAsnLysValThrGluMetLeuAspArgTyr
SerIleGlyGlnHisGluArgValProLeuGlyPheGlnGluIleLys
SerGlyProGluLysGlnArgGlnGlu;
GlyProGlyAlaProPheAsnLysValThrGluMetLeuAspArgTyr
SerIleGlyGlnHisGluArgValProLeuGlyPheGlnGluIleLys
SerGlyProGluLysGlnArgGlnGlu;
and other similar peptide sequences in which the terminal residues are varied, and in which the 6^{th}-27^{th} residues are ordered randomly.

15. A bioactive peptide composition comprising a peptide composition consisting essentially of a mixture of:
serum;
albumin;
casein;
peptone;
digestive enzyme; and
base;
wherein said peptide composition includes peptide bands which are stainable with Coomassie Blue, and which are not stainable with silver ions during SDS-PAGE electrophoresis analysis.


SEQUENCE LISTING (12 sequences)

SEQ ID NO: 1 - protein, 27 aa, Artificial Sequence
  Synthesized
GEPAPVLGQAVFPSTLEKRYIDRGQNK

SEQ ID NO: 2 - protein, 27 aa, Artificial Sequence
  Synthesized
GEPAPVLGQAVFPSTLEKRYIDRGQNE

SEQ ID NO: 3 - protein, 27 aa, Artificial Sequence
  Synthesized
GEPAPVLGQAVFPSTLEKRYIDRGQKN

SEQ ID NO: 4 - protein, 27 aa, Artificial Sequence
  Synthesized
AEPGPVLGQAVFPSTLEKRYIDRGQNE

SEQ ID NO: 5 - protein, 41 aa, Artificial Sequence
  Synthesized
APGAPFNKVTEMLDRYSIGQHERVPLGFQEIKSGPEKQRQL

SEQ ID NO: 6 - protein, 41 aa, Artificial Sequence
  Synthesized
APGAPFNKVTEMLDRYSIGQHERVPLGFQEIKSGPEKQRQK

SEQ ID NO: 7 - protein, 41 aa, Artificial Sequence
  Synthesized
APGAPFNKVTEMLDRYSIGQHERVPLGFQEIKSGPEKQRQE

SEQ ID NO: 8 - protein, 41 aa, Artificial Sequence
  Synthesized
GPGAPFNKVTEMLDRYSIGQHERVPLGFQEIKSGPEKQRQE

SEQ ID NO: 9 - protein, 59 aa, Artificial Sequence
  Synthesized
GEPGADKSTPMFVLQHRPIETGSKYEADRFQPVAIGNKRQALQGPVERLEKPGFYLPQK

SEQ ID NO: 10 - protein, 59 aa, Artificial Sequence
  Synthesized
GEPGADKSTPMFVLQHRPIETGSKYEADRFQPVAIGNKRQALQGPVERLEKPGFYLPQE

SEQ ID NO: 11 - protein, 59 aa, Artificial Sequence
  Synthesized
GEPGADKSTPMFVLQHRPIETGSKYEADRFQPVAIGNKRQALQGPVERLEKPGFYLPQN

SEQ ID NO: 12 - protein, 59 aa, Artificial Sequence
  Synthesized
AEPGADKSTPMFVLQHRPIETGSKYEADRFQPVAIGNKRQALQGPVERLEKPGFYLPQN